(19) United States
(12) Patent Application Publication
(10) Pub. No.: US 2008/0213286 A1
(43) Pub. Date: Sep. 4, 2008

(54) Mam Domain Containing Protein

(75) Inventors: Stephen Noel Fitzgerald, Cambridge (GB); Richard Joseph Fagan, London (GB); Christine Power, Thoiry (FR); Melanie Yorke, Confignon (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Ares Trading S.A., Aubonne (CH)

(21) Appl. No.: 11/577,443

(22) PCT Filed: Oct. 18, 2005
(86) PCT No.: PCT/GB2005/004032

§ 371 Date: Mar. 3, 2008

(30) Foreign Application Priority Data
Oct. 18, 2004 (GB) 0423126.2

(51) Int. Cl. A61K 39/395; C07K 14/00; C12N 15/11; C12N 9/00; A61K 31/70; A61K 38/00; A61K 39/00; A61P 43/00; C12Q 1/68; G01N 33/53; A01K 67/027; C12N 15/00; C12N 5/06; C07K 16/18

(52) U.S. Cl. 424/172.1; 530/350; 536/23.5; 435/183; 514/44; 514/12; 424/184.1; 435/6; 435/7.1; 800/18; 435/320.1; 435/325; 530/387.9; 435/7.21

(57) ABSTRACT

This invention relates to a novel protein, termed INSP152, herein identified as a secreted protein, in particular, as a MAM domain containing protein and to the use of this protein and nucleic acid sequence from the encoding gene in the diagnosis, prevention and treatment.

This invention relates to a novel protein, termed INSP152, herein identified as a secreted protein, in particular, as a MAM domain containing protein and to the use of this protein and nucleic acid sequence from the encoding gene in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### Introduction

### Secreted Proteins

The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors. Description of some of the properties of these proteins follows.

### MAM Domain Containing Proteins

MAM (meprin, A-5 protein, receptor protein-tyrosine phosphatase μ) domains are comprised of about 160 amino acids. They are present in transmembrane proteins such as the meprins and receptor protein-tyrosine phosphatases, where they appear to function in cell/cell interactions.

Meprins belong to the “astacin family” of metalloendopeptidases and the “metzincin superfamily”. They are multidomain, oligomeric proteases that are secreted or highly expressed in mammalian brush-border membranes of the intestine or kidney. The meprins are complex and structurally unique proteases: homo- or heterotetrameric glycoproteins that contain astacin-like catalytic domains and disulfide-bridged dimers. They are capable of degrading proteins such as collagen and gelatin, hormones such as parathyroid hormone, luteinizing hormone-releasing hormone, and melanocyte-stimulating hormone, and small peptides such as bradykinin, angiotensins, and gastrin.

All receptor-like protein tyrosine phosphatases (RPTPs) type II contain a MAM domain. These proteins regulate many important cellular functions including signal transduction, growth, differentiation, cell adhesion and axon guidance. MAM domains have also been shown to be linked to sperm adhesion. The domain may therefore play a role in gamete recognition and/or signalling.

Increasing knowledge of these domains is therefore of extreme importance in increasing the understanding of the underlying pathways that lead to the disease states and associated disease states mentioned above, and in developing more effective gene and/or drug therapies to treat these disorders.

### THE INVENTION

The invention is based on the discovery that the INSP152 polypeptide is a MAM domain containing protein. In particular, the invention is based on the surprising findings that polypeptides of the present invention:
a) show unexpected restricted expression in the small intestine, in lupus and in Crohn's disease biopsy samples derived from ileal biopsies and
b) are potent inhibitors of lymphocytes, preferably of T cells and
c) are CTLA4-Ig-like polypetides.

In one embodiment of the first aspect of the invention, there is provided a polypeptide which:
(i) comprises the amino acid sequence as recited in SEQ ID NO:2;
(ii) comprises the amino acid sequence as recited in SEQ ID NO: 4;
(iii) is a fragment thereof which functions as a secreted protein or has an antigenic determinant in common with the polypeptides of (i); or
(iv) is a functional equivalent of (i) or (ii).

Preferably, the polypeptide according to this first aspect of the invention is selected from the group consisting of:
(i) the amino acid sequence recited in SEQ ID NO: 6;
(ii) the amino acid sequence recited in SEQ ID NO: 8;
(iii) the amino acid sequence recited in SEQ ID NO: 10;
(iv) the amino acid sequence recited in SEQ ID NO: 12;
(v) the amino acid sequence recited in SEQ ID NO: 14;
(vi) the amino acid sequence recited in SEQ ID NO: 16; or
(vii) the amino acid sequence recited in SEQ ID NO: 18;
(viii) is a fragment thereof which functions as a secreted protein or has an antigenic determinant in common with the polypeptides of (i) to (vii); or
(ix) is a functional equivalent of parts (i) to (vii).

The polypeptide having the sequence recited in SEQ ID NO: 6 is referred to hereafter as the “cloned INSP152-D1-SV1 polypeptide”. The polypeptide having the sequence recited in SEQ ID NO: 12 is referred to hereafter as the “extracellular INSP152-SV1 polypeptide”. The polypeptide having the sequence recited in SEQ ID NO: 18 is referred to hereafter as the “INSP152-SV1 polypeptide”. The polypeptide having the sequence recited in SEQ ID NO: 20 is referred to hereafter as the “INSP152-D1 fragment polypeptide”. The polypeptide having the sequence recited in SEQ ID NO: 22 is referred to hereafter as the “full cloned INSP152-D1-SV1 polypeptide”. The polypeptide having the sequence recited in SEQ ID NO: 24 is referred to hereafter as the “extracellular INSP152 polypeptide”. The polypeptide having the sequence recited in SEQ ID NO: 26 is referred to hereafter as the “full INSP152 polypeptide”. The polypeptide having the sequence recited in SEQ ID NO: 28 is referred to hereafter as the “full INSP152-SV1 polypeptide”.

A polypeptide according to the first aspect of the invention may comprise a signal peptide and/or a histidine tag. Preferably the histidine tag is found at the C-terminal of the polypeptide. Preferably the histidine tag comprises 1-10 histidine residues (e.g. 1, 2, 3, 4, 5, 6, 7, 8, 9 or 10 residues). More preferably, the histidine tag comprises 6 histidine residues.

Although the Applicant does not wish to be bound by this theory, it is postulated that the first 19 amino acids of the INSP152 polypeptide form a signal peptide.

The mature INSP152-D1 fragment polypeptide sequence without this postulated signal sequence is recited in SEQ ID NO: 2. The cloned INSP152-D1-SV1 polypeptide sequence without this postulated signal sequence is recited in SEQ ID NO: 4. The extracellular INSP152 polypeptide sequence without this postulated signal sequence is recited in SEQ ID NO: 8. The extracellular INSP152-SV1 polypeptide sequence without this postulated signal sequence is recited in SEQ ID NO: 10. The INSP152 polypeptide sequences without this postulated signal sequence is recited in SEQ ID NO: 14. The INSP152-SV1 polypeptide sequence without this postulated signal sequence is recited in SEQ ID NO: 16.

The polypeptide having the sequence recited in SEQ ID NO: 2 is referred to hereafter as “mature INSP152-D1 fragment polypeptide”. The polypeptide having the sequence recited in SEQ ID NO: 4 is referred to hereafter as “mature cloned INSP152-D1-SV1 polypeptide”. The polypeptide having the sequence recited in SEQ ID NO: 8 is referred to hereafter as “mature extracellular INSP152 polypeptide”. The polypeptide having the sequence recited in SEQ ID NO: 10 is referred to hereafter as “mature extracellular INSP152-SV1 polypeptide”. The polypeptide having the sequence recited in SEQ ID NO: 14 is referred to hereafter as “mature INSP152 polypeptide”. The polypeptide having the sequence recited in SEQ ID NO: 16 is referred to hereafter as “mature INSP152-SV1 polypeptide”.

The term “INSP152 polypeptides” as used herein includes polypeptides comprising the mature INSP152-D1 fragment polypeptide, the mature cloned INSP152-D1-SV1 polypeptide, the cloned INSP152-D1-SV1 polypeptide, the mature extracellular INSP152 polypeptide, the mature extracellular INSP152-SV1 polypeptide, the extracellular INSP152-SV1 polypeptide, the mature INSP152 polypeptide, the mature INSP152-SV1 polypeptide, the INSP152-SV1 polypeptide, the INSP152-D1 fragment polypeptide, the full cloned INSP152-D1-SV1 polypeptide, the extracellular INSP152 polypeptide, the full INSP152 polypeptide and the full INSP152-SV1 polypeptide.

The term “MAM domain containing protein” refers to a molecule containing at least one MAM domain.

Preferably, the “MAM domain containing protein” may be a molecule containing a MAM domain detected with an e-value lower than 0.1, 0.01, 0.001, 0.0001, 0.0002, 0.00001, 0.000001 or 0.0000001.

Preferably, a polypeptide according to any one of the above-described aspects of the invention functions as a MAM domain containing and/or LDL domain containing polypeptide.

By “functions as a MAM domain containing protein” we refer to polypeptides that comprise amino acid sequence or structural features that can be identified as conserved features within the polypeptides of the MAM domain containing protein family, such that the polypeptide's interaction with protease is maintained intact. In particular, we refer to the presence of cysteine residues in specific positions within the polypeptide that allow the formation of disulphide bonds.

The so-called MAM domain is an approximately 170 amino acid domain that has been recognised in the extracellular region of functionally diverse proteins. These proteins have a modular, receptor-like architecture comprising a signal peptide, an N-terminal extracellular domain, a single transmembrane domain and an intracellular domain. Such proteins include meprin (a cell surface glycoprotein); A5 antigen (a developmentally-regulated cell surface protein); and receptor-like tyrosine protein phosphatase. The MAM domain is thought to have an adhesive function. It contains 4 conserved cysteine residues, which probably form disulphide bridges. Mutations in the meprin MAM domain affect noncovalent associations within meprin oligomers. In receptor tyrosine phosphatase 1-like molecules the MAM domain is important for homophilic cell-cell interactions.

### MAM Containing Proteins Include:

Meprin. This cell surface glycoprotein contains a zinc-metalloprotease domain capable of degrading a variety of polypeptides. Meprin is composed of two structurally related subunits (alpha and beta) that form homo- or heterotetramers by the non-covalent association of two disulfide-linked dimers. In both subunits, the MAM domain is located after the catalytic domain. The MAM domain in meprin is involved in oligomerization.

Neuropilin (A5 antigen), a calcium-independent cell adhesion molecule that functions during the formation of certain neuronal circuits. The sequence contains 2 CUB domains (2 FA58C domains) and a MAM domain.

Receptor-like tyrosine protein phosphatases Mu, Kappa and PCP-2 (EC 3.1.3.48). These PTPases have an extracellular region which consists of a MAM domain followed by an Ig-like domain and four fibronectin-type III domains. The family of PTPs consists of soluble and receptor-like PTPs (RPTPs). The extracellular region of PTPμ promotes cell-cell aggregation (the MAM domain of PTPμ is a homophilic binding molecule of the extracellular region). Homophilic binding in the ectodomains of PTPκ and PTPλ strongly suggests involvement of RPTPs in signal transduction through cell-to-cell contact. The MAM domain of PTPμ has homophilic binding properties through four conserved cysteine residues forming two intramolecular disulfide bridges (PTPμ cis (lateral) interactions) (see Cismasiu et al. J Biol Chem. 2004 279(26):26922-31). RPTPs have been involved during development, in axon growth and guidance, neurite outgrowth, as cell adhesion molecules (CAMs) and cell adhesion mediators, cell signaling, in target selection and recognition by retinal axon, and hematopoietic cell differentiation (see Beltran and Bixby, Front Biosci. 2003 8:d87-99.). CAMs are involved in homoestasis, immune response, inflammation, axonogenesis, and embryogenesis. Disruption of CAMs results in the onset of diseases such as Penphigous vulgaris, Leukocyte adhesion deficiency-1 and -2, Glanzmann thrombasthenia, atherosclerosis, diabetic vasculopathy and cancer metastasis.

Vertebrate enteropeptidase (EC 3.4.21.9), a type II membrane protein of the intestinal brush border, which activates trypsinogen. It consists at least of a catalytic light chain and a multidomain heavy chain which has 2 LDL receptor class A domains, a MAM domain, a SRCR domain and a CUB domain.

Apical endosomal glycoprotein from rat, a protein probably involved in the sorting and selective transport of receptors and ligands across polarized epithelia. This protein contains 6 MAM domains. FIG. 9 shows the structural homology between INSP152 and apical endosomal glycoprotein.

Xenopus laevis thyroid hormone induced protein B. This protein contains 4 MAM domains. An amino acid alignment between Xenopus laevis thyroid hormone induced protein B and INSP152 is shown in FIG. 2.

Pig zonadhesin, a protein that binds in a species-specific manner to the zona pellucida of the egg.

A polypeptide according to the invention may also function as an LDL domain containing polypeptide. By “functions as an LDL domain containing protein” we refer to polypeptides that comprise amino acid sequence or structural features that can be identified as conserved features within the polypeptides of the LDL domain containing protein family, such that the polypeptide's interaction with its biological protein partners is maintained. In particular, we refer to the presence of cysteine residues in specific positions within the polypeptide that allow the formation of disulphide bonds.

The cysteine-rich repeat in the low-density lipoprotein (LDL) receptor plays a central role in mammalian cholesterol metabolism. The N-terminal type A repeats in LDL receptor bind the lipoproteins. Other homologous domains occur in related receptors, including the very low-density lipoprotein receptor and the LDL receptor-related protein/alpha 2-macroglobulin receptor, and in proteins which are functionally unrelated, such as the C9 component of complement. Mutations in the LDL receptor gene cause familial hypercholesterolemia.

The LDL-receptor class A domain contains 6 disulfide-bound cysteines and a highly conserved cluster of negatively charged amino acids, of which many are clustered on one face of the module. A schematic representation of this domain is shown here:

```
  +---------------------+ +--------------------------------+
  | | | |
-CxxxxxxxxxxxxCxxxxxxxxCxxxxxxxxCxxxxxxxxxxCxxxxxxxxxxxxxxxxxxxxxC-
                            | |
                            +----------------------------+
‘C’: conserved cysteine involved in a disulfide bond.
‘x’: any residue.
```

In LDL-receptors the class A domains form the binding site for LDL and calcium. The acidic residues between the fourth and sixth cysteines are important for high-affinity binding of positively charged sequences in LDLR's ligands. The repeat has been shown to consist of a beta-hairpin structure followed by a series of beta turns. The binding of calcium seems to induce no significant conformational change.

Following these repeats is a 350 residue domain that resembles part of the epidermal growth factor (EGF) precursor (which has been identified in INSP152, see FIG. 9).

Similar domains have been found in other extracellular and membrane proteins which are listed below:

Vertebrate very low density lipoprotein (VLDL) receptor, which binds and transports VLDL. Its extracellular domain is composed of 8 LDLRA domains, 3 EGF-like domains and 6 LDL-receptor class B domains (LDLRB).

Vertebrate low-density lipoprotein receptor-related protein 1 (LRP1), which may act as a receptor for the endocytosis of extracellular ligands. LRP1 contains 31 LDLRA domains and 22 EGF-like domains.

Vertebrate low-density lipoprotein receptor-related protein 2 (LRP2) (also known as gp330 or megalin). LRP2 contains 36 LDLRA domains and 17 EGF-like domains.

A LRP-homolog from Caenorhabditis elegans, which contains 35 LDLRA domains and 17 EGF-like domains.

Drosophila putative vitellogenin receptor, with 13 copies of LDLRA domains and 17 EGF-like repeats.

Complement factor I, which is responsible for cleaving the alpha-chains of C4b and C3b. It consists of a FIMAC domain (Factor I/MAC proteins C6/C7), a scavenger receptor-like domain, 2 copies of LDLRA and a C-terminal serine protease domain.

Complement components C6, C7, C8 and C9. They contain each one LDLRA domain.

Perlecan, a large multidomain basement membrane heparan sulfate proteoglycan composed of 4 LDLRA domains, 3 LamB domains, 12 laminin EGF-like domains, 14-21 IG-like domains, 3 LamG domains, and 4 EGF-like domains. A similar but shorter proteoglycan (UNC52) is found in Caenorhabditis elegans which has 3 repeats of LDLRA.

Invertebrate giant extracellular hemoglobin linker chains, which allow heme-containing chains to construct giant hemoglobin (1 LDLRA domain).

G-protein coupled receptor Grl101 of the snail Lymnaea stagnalis, which might directly transduce signals carried by large extracellular proteins.

Vertebrate enterokinase (EC 3.4.21.9), a type II membrane protein of the intestinal brush border, which activates trypsinogen. It consists at least of a catalytic light chain and a multidomain heavy chain which has 2 LDLRA, a MAM domain, a SRCR domain and a CUB domain.

Human autosomal dominant polycystic kidney disease protein 1 (PKD1), which is involved in adhesive protein-protein and protein-carbohydrate interactions. The potential calcium-binding site of its single LDLRA domain is missing.

Vertebrate integral membrane protein DGCR2/IDD, a potential adhesion receptor with 1 LDLRA domain, a C-type lectin and a VWFC domain.

Drosophila serine protease nudel (EC 3.4.21.), which is involved in the induction of dorsoventral polarity of the embryo. It has 11 LDLRA domains, 3 of which miss the first disulfide bond (C1-C3).

Avian subgroup A rous sarcoma virus receptor (1 copy of LDLRA).

Bovine Sco-spondin, which is secreted by the subcommissural organ in embryos and is involved in the modulation of neuronal aggregation. It contains at least 2 EGF-like domains and 3 LDLRA domains.

Preferably, the activity of a polypeptide of the present invention can be confirmed in at least one of the following assays:
a) in the mouse model of inflammatory bowel disease induced by dextran sulphate sodium (DSS) as described in Okayasu et al. A novel method in the induction of reliable experimental acute and chronic ulcerative colitis in mice. Gastroenterology 1990. Vol. 98, pp. 694-702), or
b) in the in vitro and in vivo assays as well as animal models of inflammatory bowel diseases as reviewed by Borm and Gouma (Drug Discovery Today: Disease Models; Vol. 1, No. 4, 2004, pp. 437-443), or
c) in models of systemic lupus erythematosus as reviewed by Rigby and Vyse (Drug Discovery Today: Disease Models; Vol. 1, No. 4, 2004, pp. 445-449), or
d) in the human MLR assay as described in Example 7.

An “antigenic determinant” of the present invention may be a part of a polypeptide of the present invention, which binds to an antibody-combining site or to a T-cell receptor (TCR). Alternatively, an “antigenic determinant” may be a site on the surface of a polypeptide of the present invention to which a single antibody molecule binds. Generally an antigen has several or many different antigenic determinants and reacts with antibodies of many different specificities. Preferably, the antibody is immunospecific to a polypeptide of the invention. Preferably, the antibody is immunospecific to a polypeptide of the invention, which is not part of a fusion protein. Preferably, the antibody is immunospecific to any one of the INSP152 polypeptides described herein, such as INSP152 or a fragment thereof. Antigenic determinants usually consist of chemically active surface groupings of molecules, such as amino acids or sugar side chains, and can have specific three dimensional structural characteristics, as well as specific charge characteristics. Preferably, the “antigenic determinant” refers to a particular chemical group on a polypeptide of the present invention that is antigenic, i.e. that elicit a specific immune response.

The polypeptides Q5VYJ5_HUMAN and CAH71834 (AL354695), and their encoding nucleic acid sequences are specifically excluded from the scope of this invention.

In a second aspect, the invention provides a purified nucleic acid molecule which encodes a polypeptide of the first aspect of the invention.

The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “purified nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. In a preferred embodiment, genomic DNA are specifically excluded from the scope of the invention. Preferably, genomic DNA larger than 10 kbp (kilo base pairs), 50 kbp, 100 kbp, 150 kbp, 200 kbp, 250 kbp or 300 kbp are specifically excluded from the scope of the invention. Preferably, the “purified nucleic acid molecule” consists of cDNA only.

Preferably, the purified nucleic acid molecule comprises the nucleic acid sequence as recited in SEQ ID NO: 1 (encoding the mature INSP152-D1 fragment polynucleotide sequence), SEQ ID NO: 3 (encoding the mature cloned INSP152-D1-SV1 polynucleotide sequence), SEQ ID NO: 5 (encoding the cloned INSP152-D1-SV1 polynucleotide sequence), SEQ ID NO: 7 (encoding the mature extracellular INSP152 polynucleotide sequence), SEQ ID NO: 9 (encoding the mature extracellular INSP152-SV1 polynucleotide sequence), SEQ ID NO: 11 (encoding the extracellular INSP152-SV1 polynucleotide sequence), SEQ ID NO: 13 (encoding the mature INSP152 polynucleotide sequence), SEQ ID NO: 15 (encoding the mature INSP152-SV1 polynucleotide sequence), SEQ ID NO: 17 (encoding the INSP152-SV1 polynucleotide sequence), SEQ ID NO: 19 (encoding the INSP152-D1 fragment polynucleotide sequence), SEQ ID NO: 21 (encoding the cloned INSP152-D1-SV1 polynucleotide sequence), SEQ ID NO: 23 (encoding the extracellular INSP152 polynucleotide sequence), SEQ ID NO: 25 (encoding the full INSP152 polynucleotide sequence) and/or SEQ ID NO: 27 (encoding the full INSP152-SV1 polynucleotide sequence) or is a redundant equivalent or fragment of any one of these sequences.

The invention further provides that the purified nucleic acid molecule consists of the nucleic acid sequence as recited in SEQ ID NO: 1 (encoding the mature INSP152-D1 fragment polynucleotide sequence), SEQ ID NO: 3 (encoding the mature cloned INSP152-D1-SV1 polynucleotide sequence), SEQ ID NO: 5 (encoding the cloned INSP152-D1-SV1 polynucleotide sequence), SEQ ID NO: 7 (encoding the mature extracellular INSP152 polynucleotide sequence), SEQ ID NO: 9 (encoding the mature extracellular INSP152-SV1 polynucleotide sequence), SEQ ID NO: 11 (encoding the extracellular INSP152-SV1 polynucleotide sequence), SEQ ID NO: 13 (encoding the mature INSP152 polynucleotide sequence), SEQ ID NO: 15 (encoding the mature INSP152-SV1 polynucleotide sequence), SEQ ID NO: 17 (encoding the INSP152-SV1 polynucleotide sequence), SEQ ID NO: 19 (encoding the INSP152-D1 fragment polynucleotide sequence), SEQ ID NO: 21 (encoding the cloned INSP152-D1-SV1 polynucleotide sequence), SEQ ID NO: 23 (encoding the extracellular INSP152 polynucleotide sequence), SEQ ID NO: 25 (encoding the full INSP152 polynucleotide sequence) and/or SEQ ID NO: 27 (encoding the full INSP152-SV1 polynucleotide sequence) or is a redundant equivalent or fragment of any one of these sequences.

In a third aspect, the invention provides a purified nucleic acid molecule which hybridizes under high stringency conditions with a nucleic acid molecule of the second aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to protein members of the MAM domain containing protein family of the first aspect of the invention. Preferably, the ligand inhibits the function of a polypeptide of the first aspect of the invention which is a MAM domain containing protein. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention. Such compounds may be identified using the assays and screening methods disclosed herein.

A compound of the seventh aspect of the invention may either increase (agonise) or decrease (antagonise) the level of expression of the gene or the activity of the polypeptide. Importantly, the identification of the function of the INSP152 polypeptides allows for the design of screening methods capable of identifying compounds that are effective in the treatment and/or diagnosis of disease. Ligands and compounds according to the sixth and seventh aspects of the invention may be identified using such methods. These methods are included as aspects of the present invention.

Another aspect of this invention resides in the use of an INSP152 gene or polypeptide as a target for the screening of candidate drug modulators, particularly candidate drugs active against MAM domain containing protein related disorders.

A further aspect of this invention resides in methods of screening of compounds for therapy of MAM domain containing protein related disorders, comprising determining the ability of a compound to bind to an INSP152 gene or polypeptide, or a fragment thereof.

A further aspect of this invention resides in methods of screening of compounds for therapy of MAM domain containing protein related disorders, comprising testing for modulation of the activity of an INSP152 gene or polypeptide, or a fragment thereof.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which MAM domain containing proteins are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anaemia, sepsis, shock, reperfusion injury, stroke and ischaemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolaemia, hyperlipidaemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions. These molecules may also be used in the manufacture of a medicament for the treatment of such diseases. These molecules may also be used in contraception or for the treatment of reproductive disorders including infertility.

Most particularly, diseases in which MAM domain containing proteins are implicated are selected from inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea. As described herein, results of a Taqman analysis show unexpected restricted expression of INSP152 in small intestine (see Table 5 and FIG. 10), in a lupus sample (see Table 6 and FIG. 11), and in 12/22 Crohn's disease biopsy samples (see Table 7 and FIG. 12). This specific pattern of expression leads to the conclusion of the involvement of INSP152, particularly INSP152-D1-SV1, in diseases of the small intestine, including Crohn's disease, small bowel disease, inflammatory bowel disease, lupus, celiac disease, Whipple's disease, neoplasias and diarrhea. These surprising properties characterizing the polynucleotides or the corresponding polypeptides of the present invention make them particularly suitable for the preparation of a drug or pharmaceutical composition. The polynucleotides or the corresponding polypeptides of the present invention therefore display the unexpected finding of a restricted expression in specific tissues.

Additional results (see FIG. 13) surprisingly show that the INSP152-D1-SV1 polypeptide is a potent inhibitor of lymphocytes, preferably of T cells. As such, INSP152-D1-SV1 displays similar functional activity to CTLA4-Ig. We thus infer that polypeptides according to the present invention, particularly INSP152-D1-SV1, are useful for the treatment of CTLA4-Ig related diseases. Such polypeptides are useful on their own, as a component of fusion proteins such as Fc fusion, and/or in combination with another agent.

As mentioned above, the polypeptides of the present invention are CTLA4-Ig-like polypeptides and, as such, play a role in CTLA4-related diseases. Therefore the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which CTLA4 is implicated. Such diseases include, but are not limited to, T-cell mediated disease, autoimmune thyroid disease, autoimmune disease, graves disease, autoimmunity, chronic lymphocytic thyroiditis, autoimmune hepatitis, multiple sclerosis, addisons disease, insulin-dependent diabetes mellitus, myasthenia gravis, systemic lupus erythematosus, autoimmune diabetes, rheumatoid arthritis, celiac disease, autoimmune response, thymoma, psoriasis, stage iv melanoma, encephalomyelitis, primary biliary cirrhosis, polymyositis, hypothyroidism, wegeners granulomatosis, melanoma, vitiligo, mucocutaneous lymph node syndrome, endocrine disease, inflammatory bowel disease, inflammation, alcoholic liver disease, atopy, allergic rhinitis, systemic scleroderma, uveitis, dermatomyositis, demyelination, neoplasms, arthritis, myositis, necrosis, lymphocytic leukemia, hematologic disease, virus disease, liver disease, non-insulin-dependent diabetes mellitus, cell injury, asthma, ulcerative colitis, periodontal disease, ovarian epithelial carcinoma, cirrhosis, influenza, immunologic deficiency syndrome, granuloma, hodgkin disease, malaria, diabetes mellitus, hepatitis, thrombocytopenic purpura or epidermal hyperplasia.

The polypeptides of the present invention, particularly INSP152-D1-SV1, are also useful in graft versus host disease or in blocking of allograft and xenograft rejection.

The moieties of the present invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may have particular utility in the therapy or diagnosis of disorders/diseases (the two terms are used interchangeably herein) such as cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anaemia, sepsis, shock, reperfusion injury, stroke and ischaemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolaemia, hyperlipidaemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a diagnostic method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a MAM domain containing protein. Suitable uses of the polypeptides of the invention as MAM domain containing proteins include use for fertility control and follicular development, use as part of a receptor/ligand pair and use as a diagnostic marker for a physiological or pathological condition selected from the list given above.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease, including, but not limited to, cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anaemia, sepsis, shock, reperfusion injury, stroke and ischaemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolaemia, hyperlipidaemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

The INSP152 polypeptides are MAM domain containing proteins and thus have roles in many disease states. Antagonists of the INSP152 polypeptides are of particular interest as they provide a way of modulating these disease states.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

As used herein, “functional equivalent” refers to a protein or nucleic acid molecule that possesses functional or structural characteristics that are substantially similar to a polypeptide or nucleic acid molecule of the present invention. A functional equivalent of a protein may contain modifications depending on the necessity of such modifications for the performance of a specific function. The term “functional equivalent” is intended to include the fragments, mutants, hybrids, variants, analogs, or chemical derivatives of a molecule.

Preferably, the “functional equivalent” may be a protein or nucleic acid molecule that exhibits any one or more of the functional activities of the polypeptides of the present invention.

Preferably, the “functional equivalent” may be a protein or nucleic acid molecule that displays substantially similar activity compared with INSP152 or fragments thereof in a suitable assay for the measurement of biological activity or function. Preferably, the “functional equivalent” may be a protein or nucleic acid molecule that displays identical or higher activity compared with INSP152 or fragments thereof in a suitable assay for the measurement of biological activity or function. Preferably, the “functional equivalent” may be a protein or nucleic acid molecule that displays 50%, 60%, 70%, 80%, 90%, 95%, 98%, 99%, 100% or more activity compared with INSP152 or fragments thereof in a suitable assay for the measurement of biological activity or function.

Preferably, the “functional equivalent” may be a protein or polypeptide capable of exhibiting a substantially similar in vivo or in vitro activity as the polypeptides of the invention. Preferably, the “functional equivalent” may be a protein or polypeptide capable of interacting with other cellular or extracellular molecules in a manner substantially similar to the way in which the corresponding portion of the polypeptides of the invention would. For example, a “functional equivalent” would be able, in an immunoassay, to diminish the binding of an antibody to the corresponding peptide (i.e., the peptide the amino acid sequence of which was modified to achieve the “functional equivalent”) of the polypeptide of the invention, or to the polypeptide of the invention itself, where the antibody was raised against the corresponding peptide of the polypeptide of the invention. An equimolar concentration of the functional equivalent will diminish the aforesaid binding of the corresponding peptide by at least about 5%, preferably between about 5% and 10%, more preferably between about 10% and 25%, even more preferably between about 25% and 50%, and most preferably between about 40% and 50%.

For example, functional equivalents can be fully functional or can lack function in one or more activities. Thus, in the present invention, variations can affect the function, for example, of the activities of the polypeptide that reflect its possession of a MAM domain.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

In a further preferred embodiment, the polypeptide of the invention comprising a sequence having at least 85% of homology with INSP152 is a fusion protein.

These fusion proteins can be obtained by cloning a polynucleotide encoding a polypeptide comprising a sequence having at least 85% of homology INSP152 in frame to the coding sequences for a heterologous protein sequence. A preferred fusion protein according to the invention comprises INSP152-D1-SV1. INSP152-D1-SV1 is useful on its own, as a component of fusion proteins such as Fc fusion, and/or in combination with another agent.

The term “heterologous”, when used herein, is intended to designate any polypeptide other than a human INSP152 polypeptide.

Examples of heterologous sequences, that can be comprised in the soluble fusion proteins either at N- or at C-terminus, are the following: extracellular domains of membrane-bound protein, immunoglobulin constant regions (Fc region), multimerization domains, domains of extracellular proteins, signal sequences, export sequences, or sequences allowing purification by affinity chromatography.

Many of these heterologous sequences are commercially available in expression plasmids since these sequences are commonly included in the fusion proteins in order to provide additional properties without significantly impairing the specific biological activity of the protein fused to them (Terpe K, 2003. Appl Microbiol Biotechnol, 60: 523-33). Examples of such additional properties are a longer lasting half-life in body fluids, the extracellular localization, or an easier purification procedure as allowed by the a stretch of Histidines forming the so-called “histidine tag” (Gentz et al., 1989 Proc Natl Acad Sci USA, 86: 821-4) or by the “HA” tag, an epitope derived from the influenza hemagglutinin protein (Wilson et al., 1994 Cell, 37: 767-78). If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met (SEQ ID NO: 29) introduced between the sequence of the substances of the invention and the immunoglobulin sequence. The resulting fusion protein has improved properties, such as an extended residence time in body fluids (half-life), increased specific activity, increased expression level, or the purification of the fusion protein is facilitated.

In a preferred embodiment, the protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM or IgA, for example. Moieties that possess ADCC activity are typically of the IgG1 or IgG3 isotype. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination. Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP152 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Percentage identity, as referred to herein, is as determined using BLAST version 2.1.3 using the default parameters specified by the NCBI (the National Center for Biotechnology Information; http://www.ncbi.nlm.nih.gov/) [Blosum 62 matrix; gap open penalty=11 and gap extension penalty=1].

Homologous polypeptides therefore include natural biological variants (for example, allelic variants or geographical variations within the species from which the polypeptides are derived) and mutants (such as mutants containing amino acid substitutions, insertions or deletions) of the INSP152 polypeptides. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

In one respect, the term “variant” is meant to include extended or truncated versions of the polypeptides described expictly herein.

For extended variants, it is considered likely that the MAM domain of the INSP152 polypeptides described herein will fold correctly and show biological activity if additional residues C terminal and/or N terminal of these boundaries in the polypeptide sequence are included in the polypeptide fragment. For example, an additional 5, 10, 20, 30, 40 or even 50 or more amino acid residues from INSP152 polypeptide sequences such as the INSP152-D1 fragment, the INSP152-D1-SV1 fragment, the INSP152-SV1 extracellular fragment, the INSP152-SV1 fragment itself, or from a homologous sequence, may be included at either or both the C terminal and/or N terminal of the boundaries of the MAM domain of the INSP152 polypeptides, without prejudicing the ability of the polypeptide fragment to fold correctly and exhibit biological activity. For truncated variants of the INSP152 polypeptide, one or more amino acid residues, even 5, 10, 20, 30, 40, 50 or more amino acid residues, may be deleted at either or both the C terminus or the N terminus of the MAM region of the polypeptide.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov SI and Nekrasov AN, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of, more preferred synonymous amino acids are shown in Table 1.

Specific, non-conservative mutations can be also introduced in the polypeptides of the invention with different purposes. Mutations reducing the affinity of the MAM domain containing protein may increase its ability to be reused and recycled, potentially increasing its therapeutic potency (Robinson CR, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table 2. A non-exhaustive list of amino acid derivatives also include aminoisobutyric acid (Aib), hydroxyproline (Hyp), 1,2,3,4-tetrahydro-isoquinoline-3-COOH, indoline-2-carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty DA, 2000). Techniques for the synthesis and the development of peptide mimetics, as well as non-peptide mimetics, are also well known in the art (Golebiowski A et al., 2001; Hruby V J and Balse P M, 2000; Sawyer TK, in “Structure Based Drug Design”, edited by Veerapandian P, Marcel Dekker Inc., pg. 557-663, 1997).

Typically, greater than 30% identity between two polypeptides is considered to be an indication of functional equivalence. Preferably, functionally equivalent polypeptides of the first aspect of the invention have a degree of sequence identity with the INSP152 polypeptide, or with active fragments thereof, of greater than 80%. More preferred polypeptides have degrees of identity of greater than 85%, 90%, 95%, 98% or 99%, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium™ search database may be used (see PCT application WO 01/69507) to identify polypeptides of presently-unknown function which, while having low sequence identity as compared to the INSP152 polypeptides, are predicted to be MAM domain containing proteins, by virtue of sharing significant structural homology with the INSP152 polypeptide sequence. By “significant structural homology” is meant that the Inpharmatica Genome Threader predicts two proteins to share structural homology with a certainty of 10% and above.

The polypeptides of the first aspect of the invention also include fragments of the INSP152 polypeptides and fragments of the functional equivalents of the INSP152 polypeptides, provided that those fragments are members of the MAM domain containing protein family or have an antigenic determinant in common with the INSP152 polypeptides.

As used herein, the term “fragment” refers to a polypeptide having an amino acid sequence that is the same as part, but not all, of the amino acid sequence of the INSP152 polypeptide or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant. Fragments according to the invention may be 1-100 amino acids in length, preferably, 5-50, more preferably 7-20 amino acids.

Nucleic acids according to the invention are preferably 10-6500 nucleotides in length, preferably 1000-6300 nucleotides, preferably 3000-6300, preferably 4000-6250, preferably 5000-6250 nucleotides in length. Polypeptides according to the invention are preferably 10-2500 amino acids in length, preferably 100-2400, preferably 1000-2300, preferably 2000-2250 amino acids in length.

Fragments of the full length INSP152 polypeptides may consist of combinations of neighbouring exon sequences in the INSP152 polypeptide sequences, respectively. For example, such combinations include exons 1 and 2, exons 2 and 3 or exons 1 and 3, and so on. Such fragments are included in the present invention.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known secreted proteins.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known secreted proteins such as MAM domain containing proteins.

Preferably, there is a measurable increase in the affinity for a polypeptide of the invention as compared with known MAM domain containing proteins.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology

Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al, Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is, an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26 and SEQ ID NO: 28 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26 or SEQ ID NO: 28. Such nucleic acid molecules may include, but are not limited to, the coding sequence for the mature polypeptide by itself; the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP152 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INSP152 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP152 polypeptides and to isolate cDNA and genomic clones of homologous or orthologous genes that have a high sequence similarity to the gene encoding this polypeptide.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP152 polypeptide is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989, 1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25 and SEQ ID NO: 27), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The TOPO vector and pEAK12d-PAC_INSP152-D1-SV1-6HIS-V1 vector are preferred examples of a suitable vectors for use in accordance with the aspects of this invention relating to INSP152.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptoinyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively. Also, antimetabolite, antibiotic or herbicide resistance can be used as the basis for selection; for example, dihydrofolate reductase (DHFR) that confers resistance to methotrexate (Wigler, M. et al (1980) Proc. Natl. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmuiioassay [RIA]), that include membrane, solution, or chip based technologies for the detection and/or quantification of nucleic acid or protein (see Hampton, R. et al (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp. (Cleveland, Ohio)).

Suitable reporter molecules or labels, which may be used for ease of detection, include radionucleides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

Methods for generating detectable signals in the types of assays described herein will be known to those of skill in the art. A particular example is cotransfecting a construct expressing a polypeptide according to the invention, or a fragment that is responsible for binding to target, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential target or ligand is added to the cells, it will bind the GAL4-polypeptide fusion and induce transcription of the luciferase gene. The level of the luciferase expression can be monitored by its activity using a luminescence reader (see, for example, Lehman et al. JBC 270, 12953, 1995; Pawar et al. JBC, 277, 39243, 2002).

A further preferred method for identifying an agonist or antagonist of a polypeptide of the invention comprises:
(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, by means of artificially anchoring it to the cell membrane, or by constructing a chimeric receptor being associated with a second component capable of providing a detectable signal in response to the binding of a compound to the polypeptide, with a compound to be screened under conditions to permit binding to the polypeptide; and
(c) determining whether the compound binds to and activates or inhibits the polypeptide by comparing the level of a signal generated from the interaction of the compound with the polypeptide with the level of a signal in the absence of the compound.

For example, a method such as FRET detection of a ligand bound to the polypeptide in the presence of peptide co-activators (Norris et al., Science 285, 744, 1999) might be used.

In further preferred embodiments, the general methods that are described above may further comprise conducting the identification of agonist or antagonist in the presence of labelled or unlabelled ligand for the polypeptide.

In another embodiment of the method for identifying agonist or antagonist of a polypeptide of the present invention comprises:

determining the inhibition of binding of a ligand to the polypeptide of the invention on any solid or cellular surface thereof, in the presence of a candidate compound under conditions to permit binding to the polypeptide, and determining the amount of ligand bound to the polypeptide. A compound capable of causing reduction of binding of a ligand is considered to be a competitor which may act as an agonist or antagonist. Preferably the ligand is labelled.

More particularly, a method of screening for a polypeptide antagonist or agonist compound comprises the steps of:

(a) incubating a labelled ligand with a polypeptide according to the invention on any solid support or the cell surface, or a cell membrane containing a polypeptide of the invention.
(b) measuring the amount of labelled ligand bound to the polypeptide on the solid support, whole cell or the cell membrane;
(c) adding a candidate compound to a mixture of labelled ligand and immobilized polypeptide on the solid support, the whole cell or the cell membrane of step (a) and allowing the mixture to attain equilibrium;
(d) measuring the amount of labelled ligand bound to the immobilized polypeptide or the whole cell or the cell membrane after step (c); and
(e) comparing the difference in the labelled ligand bound in step (b) and (d), such that the compound which causes the reduction in binding in step (d) is considered to be an agonist or antagonist.

The polypeptides may be found to modulate a variety of physiological and pathological processes in a dose-dependent manner in the above-described assays. Thus, the “functional equivalents” of the polypeptides of the invention include polypeptides that exhibit any of the same modulatory activities in the above-described assays in a dose-dependent manner. Although the degree of dose-dependent activity need not be identical to that of the polypeptides of the invention, preferably the “functional equivalents” will exhibit substantially similar dose-dependence in a given activity assay compared to the polypeptides of the invention.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signaling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of immobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

In another embodiment, this invention relates to the use of a INSP152 polypeptide or fragment thereof, whereby the fragment is preferably a INSP152 gene-specific fragment, for isolating or generating an agonist or stimulator of the INSP152 polypeptide for the treatment of an immune related disorder, wherein said agonist or stimulator is selected from the group consisting of:

1. a specific antibody or fragment thereof including: a) a chimeric, b) a humanized or c) a fully human antibody, as well as;
2. a bispecific or multispecific antibody,
3. a single chain (e.g. scFv) or
4. single domain antibody, or
5. a peptide- or non-peptide mimetic derived from said antibodies or
6. an antibody-mimetic such as a) an anticalin or b) a fibronectin-based binding molecule (e.g. trinectin or adnectin).

The generation of peptide- or non-peptide mimetics from antibodies is known in the art (Saragovi et al., 1991 and Saragovi et al., 1992). Anticalins are also known in the art (Vogt et al., 2004). Fibronectin-based binding molecules are described in U.S. Pat. No. 6,818,418 and WO2004029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

As mentioned above, it is envisaged that the various moieties of the invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may be useful in the therapy or diagnosis of diseases. To assess the utility of the moieties of the invention for treating or diagnosing a disease one or more of the following assays may be carried out. Note that although some of the following assays refer to the test compound as being a protein/polypeptide, a person skilled in the art will readily be able to adapt the following assays so that the other moieties of the invention may also be used as the “test compound”.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [α] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutical compositions according to the present invention may further comprise one or more additional active agent. Preferably, the additional active agent is selected from cyclophosphamide (CTX), CLTA4-Ig, rapamycin, mycophenolic acid, methylprednisone, FTY720, an LFA-1 blocking agent, ICOS-Ig, an anti-apoptotic agent, an antagonist of cytolytic T cell function, TACI-Ig or methotrexate.

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:
a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
   wherein detection of levels of the hybrid complex in the patient sample that differ from levels of the hybrid complex in the control sample is indicative of disease.

A further aspect of the invention comprises a diagnostic method comprising the steps of:
a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al.); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al.). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in which MAM domain containing proteins are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anaemia, sepsis, shock, reperfusion injury, stroke and ischaemia; neurological disorders including central nervous system disease, Alzieimer's disease, brain injury, amyotrophic lateral sclerosis, and pain; developmental disorders; metabolic disorders including diabetes mellitus, osteoporosis, hypercholesterolaemia, hyperlipidaemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions. Such kits may also be used for the detection of reproductive disorders including infertility.

Various aspects and embodiments of the present invention will now be described in more detail by way of example, with particular reference to the INSP152 polypeptides.

It will be appreciated that modification of detail may be made without departing from the scope of the invention.

### BRIEF DESCRIPTION OF THE FIGURES

FIG. 1: Top ten BLASTp hits for INSP152 polypeptide sequence (SEQ ID NO: 26) against NCBI-nr database.

FIG. 2: Alignment between INSP152 polypeptide sequence (SEQ ID NO: 26) and thyroid hormone-induced protein B precursor, a MAM domain protein.

FIG. 3: Signal peptide prediction (SignalP V2.0) for INSP152 polypeptide sequence (SEQ ID NO: 26).

FIG. 4: INSP152 (SEQ ID NO: 80) showing the 9 MAM domains and the transmembrane domain. (The 9 MAM domains predicted at ncbi-CDD are shaded. Predicted transmembrane region is in bold).

FIG. 5: DNA and protein sequence of INSP152. The predicted MAM domain 1 is underlined. Position and sense of PCR primers (INSP152-CP1/INSP152-CP2 and INSP152-CP3/INSP152-CP4) are indicated by the arrows.

FIG. 6: DNA sequence of the PCR product INSP152-D1-SV1 cloned using primers INSP152-CP3 and INSP152-CP4 and protein translation. Position and sense of PCR primers are indicated by the arrows.

FIG. 7: Alignment of the INSP152-D1-SV1 and INSP152-D1 protein sequences.

FIG. 8: Schematic representation of the splicing events leading to the assembly of INSP152-D1 and INSP152-D1-SV1 protein sequences.

FIG. 9: Schematic domain alignment of LDL and MAM domains containing proteins: rat apical endosomal glycoprotein precursor (AEGP, SwissProt Acc. No. Q63191), human UNQ3001 (SwissProt Acc. No. AAQ88785) along with the full length INSP152 and the cloned INSP152-SV1(INSP152-D1-SV1).

FIG. 10: Expression of INSP152 in major human tissues

FIG. 11: Expression of INSP152 in comparative human tissues

FIG. 12: Expression of INSP152 in diseased human colon and ileum

FIG. 13: hMLR proliferation assay results for INSP152-D1-SV1

TABLE 1

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 2

| Amino | |
|---|---|
| Acid | Synonymous Groups |
| Ser | D-Ser, Thr, D-Thr, allo-Thr, Met, D-Met, Met(O), D-Met(O), L- |
| | Cys, D-Cys |
| Arg | D-Arg, Lys, D-Lys, homo-Arg, D-homo-Arg, Met, Ile, D-.Met, |
| | D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or |
| | L-1-oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, Trans-3,4, |
| | or 5-phenylproline, AdaA, AdaG, cis-3,4, or 5-phenylproline, |
| | Bpa, D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, Ile, |
| | D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S--Me--Cys, Ile, D-Ile, Leu, D-Leu, Val, D-Val |

### EXAMPLES

### Example 1

### INSP152 Protein BLAST Results

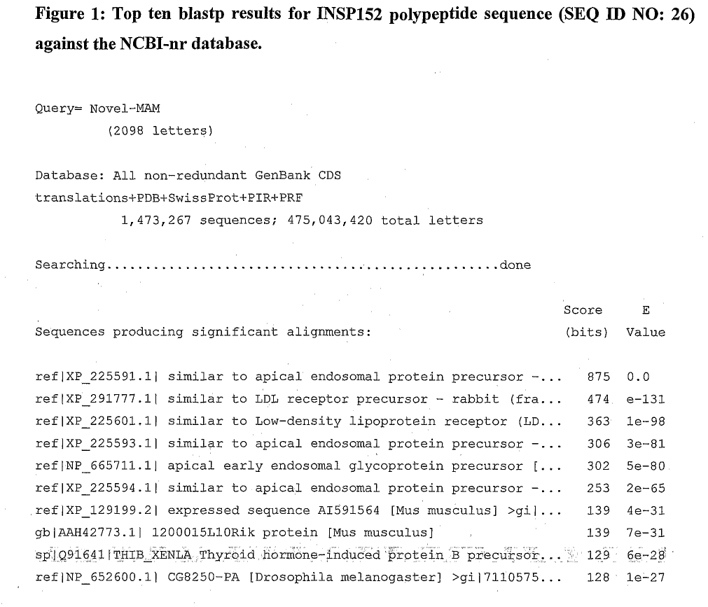

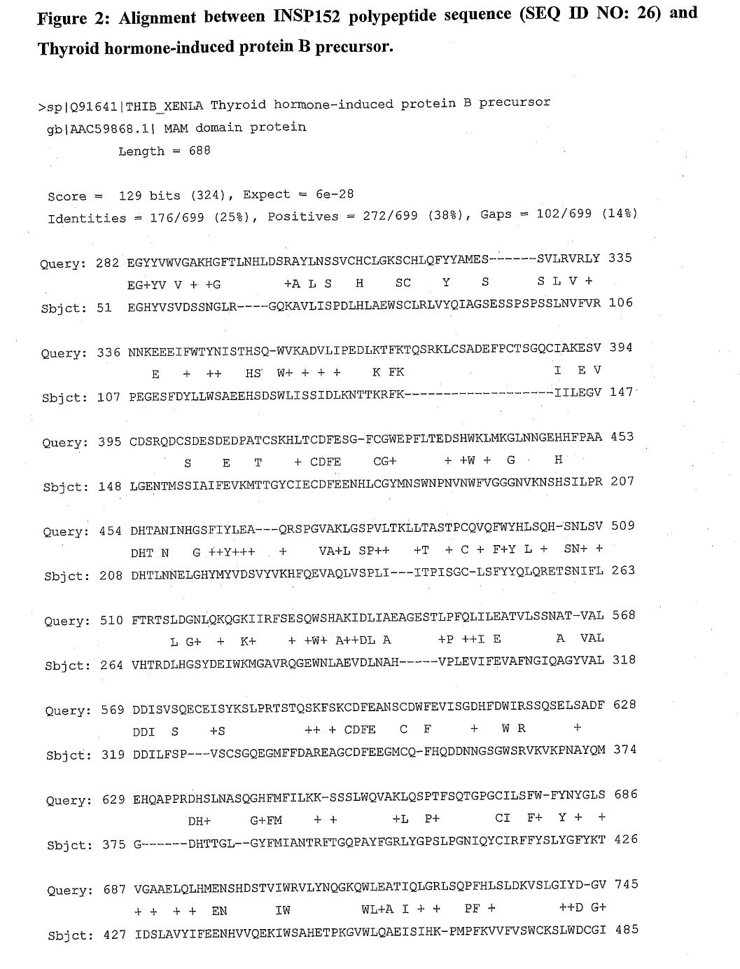

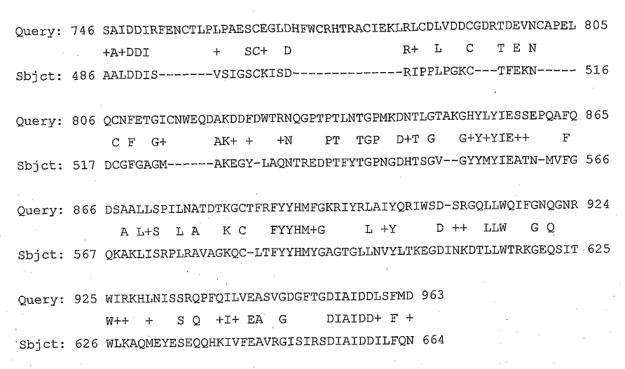

The INSP152 polypeptide sequence (SEQ ID NO: 26) was used as a protein BLAST query sequence against the NCBI non-redundant sequence database. FIG. 1 shows the top ten results for the BLAST query, and FIG. 2 shows the alignment of thyroid hormone-induced protein B precursor, a MAM domain protein with INSP152 (SEQ ID NO: 26).

### Example 2

### INSP152 Signal Sequence

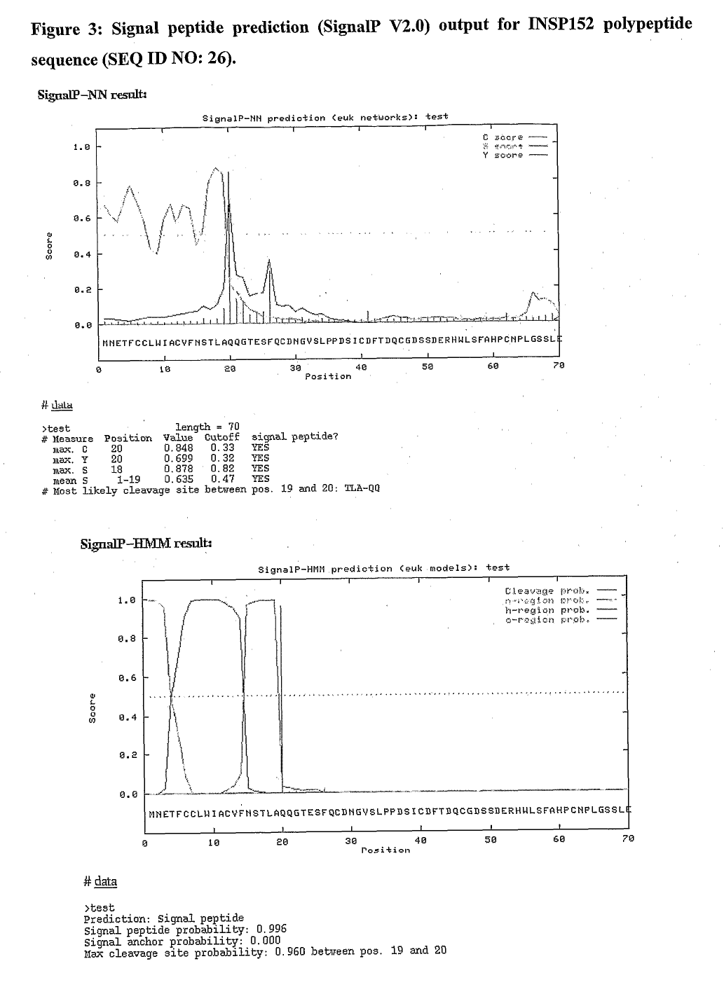

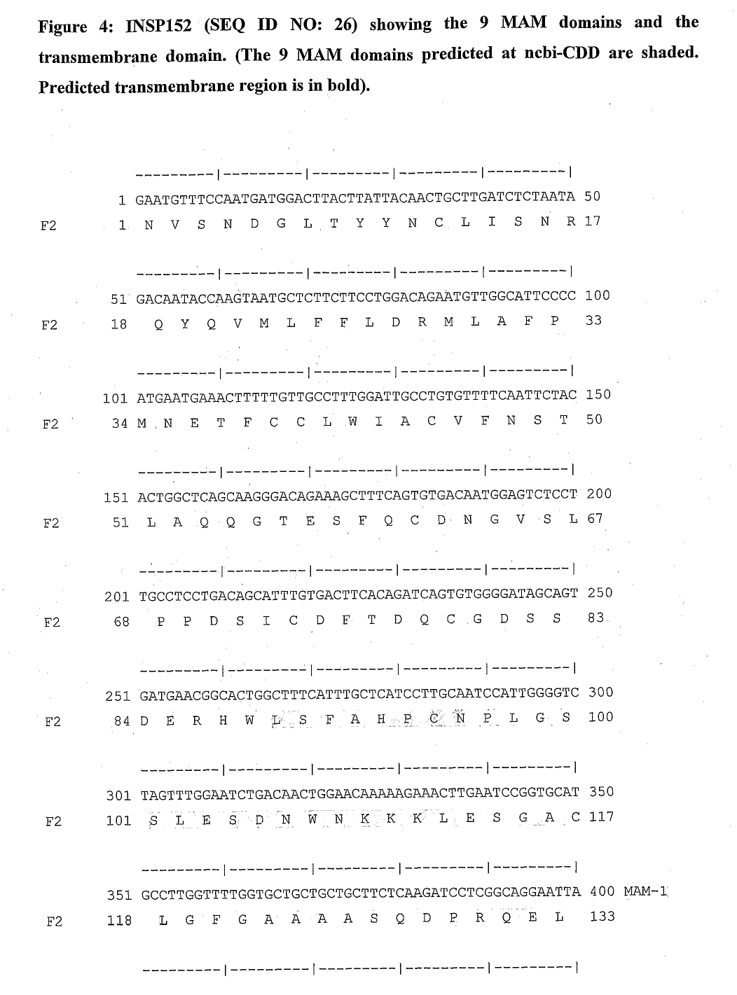

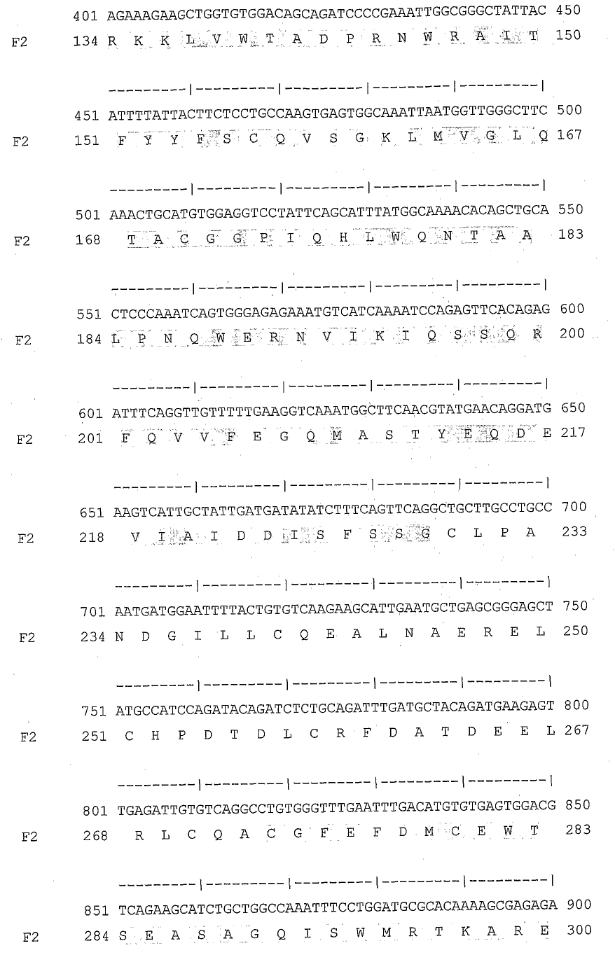

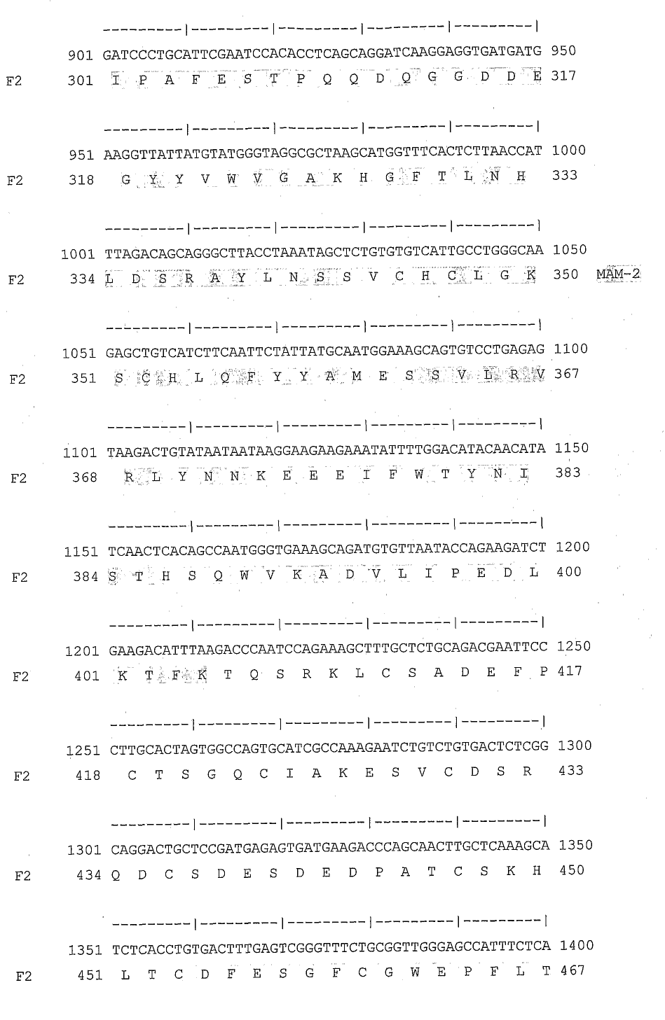

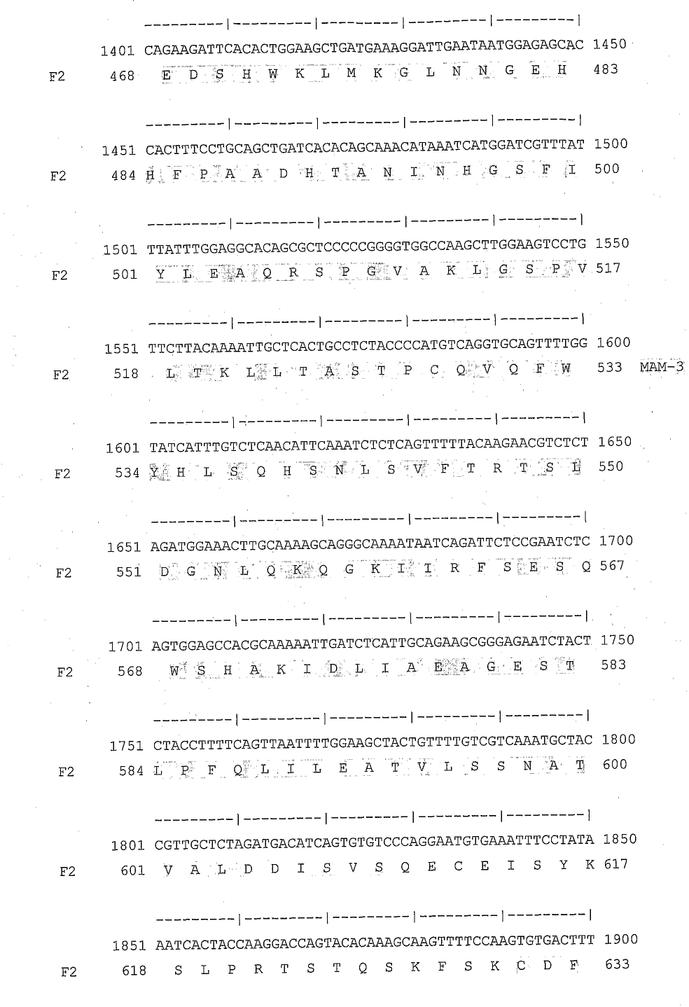

FIG. 3 show that INSP152 is predicted to possess a signal peptide at the start of the protein. As the SignalP data in FIG. 3 clearly shows, the signal peptide cleavage site is thought to be between residues 19 and 20 of the INSP152 partial polypeptide sequence (Nielsen, H. et al 1997, Protein Engineering, 10, 1-6; Nielsen, H., and Krogh, A.: Prediction of signal peptides and signal anchors by a hidden Markov model. In Proceedings of the Sixth International Conference on Intelligent Systems for Molecular Biology (ISMB 6), AAAI Press, Menlo Park, Calif., pp. 122-130 (1998)).

### Example 3

### Domain Location

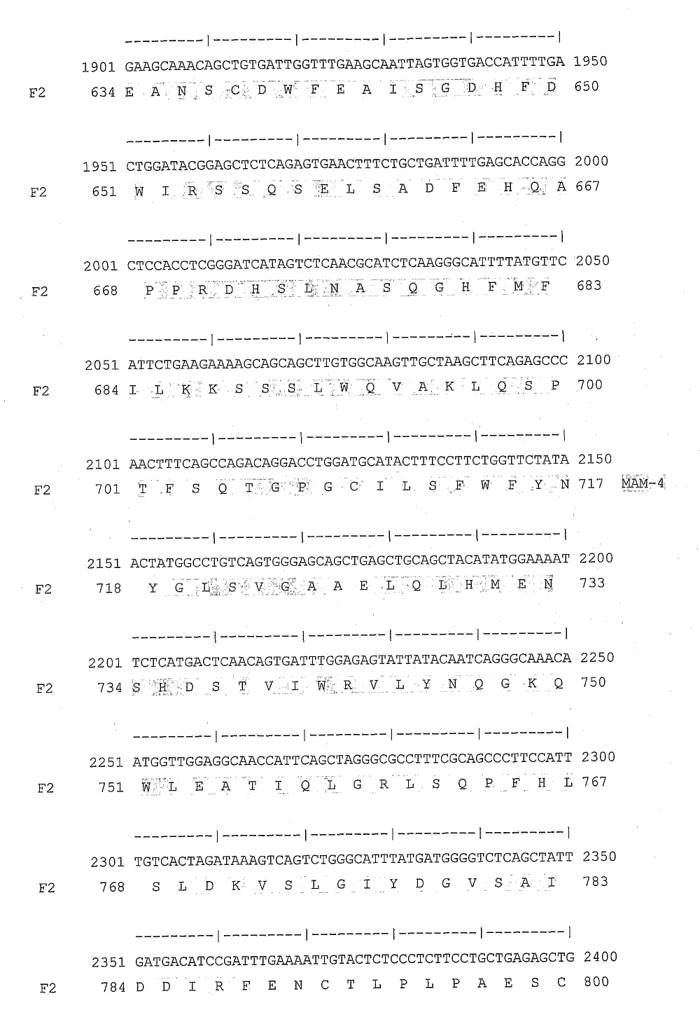

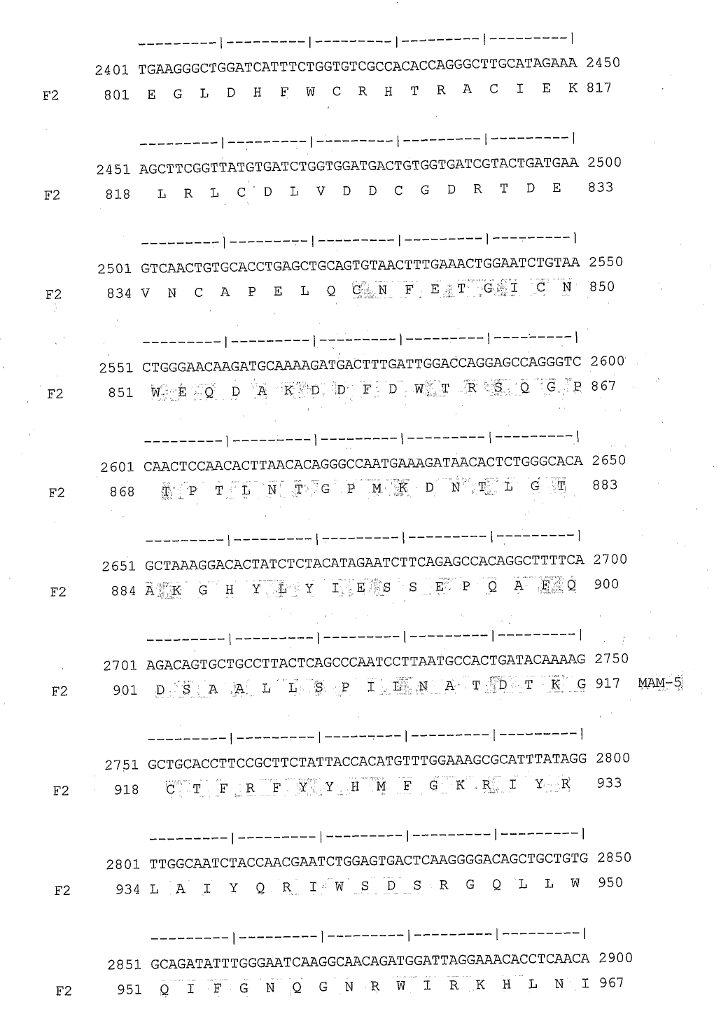

FIG. 4 shows the location of the transmembrane domain as predicted by CDD as well as the location of the 9 MAM domains.

### Example 4

### Cloning of INSP152-D1-SV1

4.1 Preparation of Human cDNA Templates

First strand cDNA was prepared from a variety of human tissue total RNA samples (Clontech, Stratagene, Ambion, Biochain Institute and in-house preparations) using Superscript II or SuperScript III RNase H—Reverse Transcriptase (Invitrogen) according to the manufacturer's protocol.

For SuperScript II: Oligo (dT)₁₅ primer (1 μl at 500 μg/ml) (Promega), 2 μg human total RNA, 1 μl 10 mM dNTP mix (10 mM each of DATP, dGTP, dCTP and dTTP at neutral pH) and sterile distilled water to a final volume of 12 μl were combined in a 1.5 ml Eppendorf tube, heated to 65° C. for 5 min and chilled on ice. The contents were collected by brief centrifugation and 4 μl of 5× First-Strand Buffer, 2 μl 0.1 M DTT, and 1 μl RnaseOUT™ Recombinant Ribonuclease Inhibitor (40 units/μl, Invitrogen) were added. The contents of the tube were mixed gently and incubated at 42° C. for 2 min, then 1 μl (200 units) of SuperScript II™ enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min.

For SuperScript III: 1 μl Oligo(dT)₂₀ primer (50 μM, Invitrogen), 2 μg human total RNA, 1 μl 10 mM dNTP mix (10 mM each of dATP, dGTP, dCTP and dTTP at neutral pH) and sterile distilled water to a final volume of 10 μl were combined in a 1.5 ml Eppendorf tube, heated to 65° C. for 5 min and then chilled on ice. For each RT reaction a cDNA synthesis mix was prepared as follows: 2μl 10×RT buffer, 4 μl 25 mM MgCl₂, 2 μl 0.1M DTT, 1 μl RNaseOUT™ (40 U/μl) and 1 μl SuperScript III™ RT enzyme were combined in a separate tube and then 10 μl of this mix added to the tube containing the RNA/primer mixture. The contents of the tube were mixed gently, collected by brief centrifugation, and incubated at 50° C. for 50 min. The reaction was terminated by incubating at 80° C. for 5 min and the reaction mixture then chilled on ice and collected by brief centrifugation. To remove RNA complementary to the cDNA, 1 μl (2 units) of E. coli RNase H (Invitrogen) was added and the reaction mixture incubated at 37° C. for 20 min.

The final 21 μl reaction mix was diluted by adding 179 μl sterile water to give a total volume of 200 μl. The RNA samples were combined into pools such that each pool contained up to five different cDNA samples. 5 μl of each cDNA pool was used as a template for PCR in a 50 μl final reaction volume and this consisted of 1 μl of each cDNA sample in that pool. This represented approximately 20 ng of each individual cDNA template.

### 4.2 INSP152-D1 Specific Cloning Primers for PCR

Two pairs of PCR primers having a length of between 18 and 30 bases were designed for amplifying a 595 bp fragment of the INSP152 predicted cds (INSP152-D1 (Domain 1), from the start of the prediction to the end of the first predicted MAM domain, using Primer Designer Software (Scientific & Educational Software, PO Box 72045, Durham, N.C. 27722-2045, USA). PCR primers were optimized to have a Tm close to 55±10° C. and a GC content of 40-60%. Primers were selected which had high selectivity for the target sequence (INSP152) with little or no none specific priming. The primers were designed to form two nested pairs such that INSP152-CP3/INSP152-CP4 primers were positioned slightly internal to INSP152-CP1/INSP152-CP2 primers.

4.3 PCR Amplification of INSP152-D1 from Human cDNA Templates

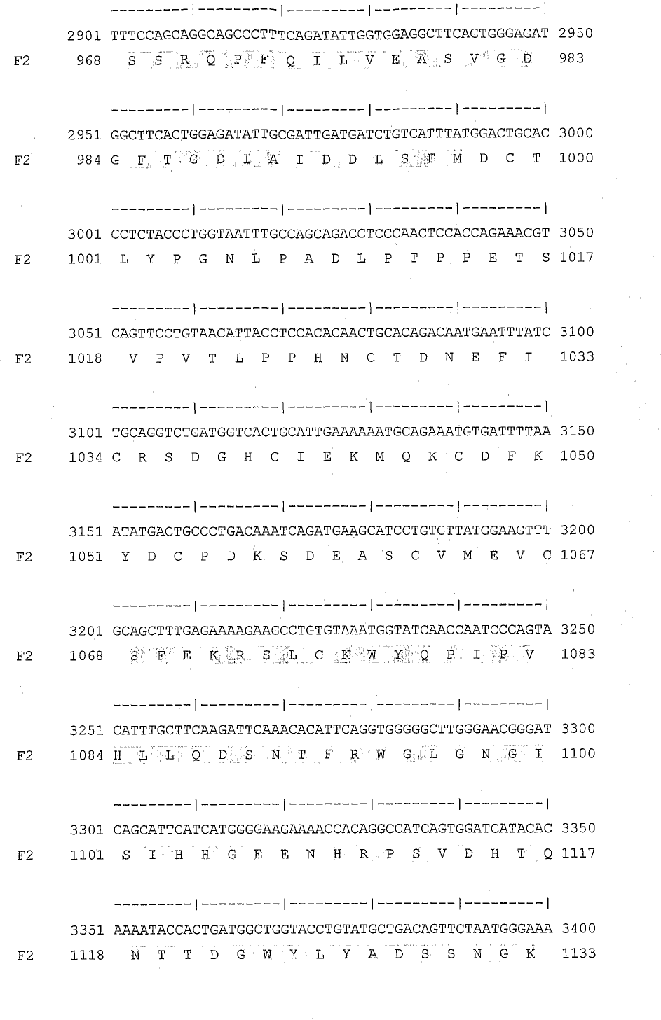

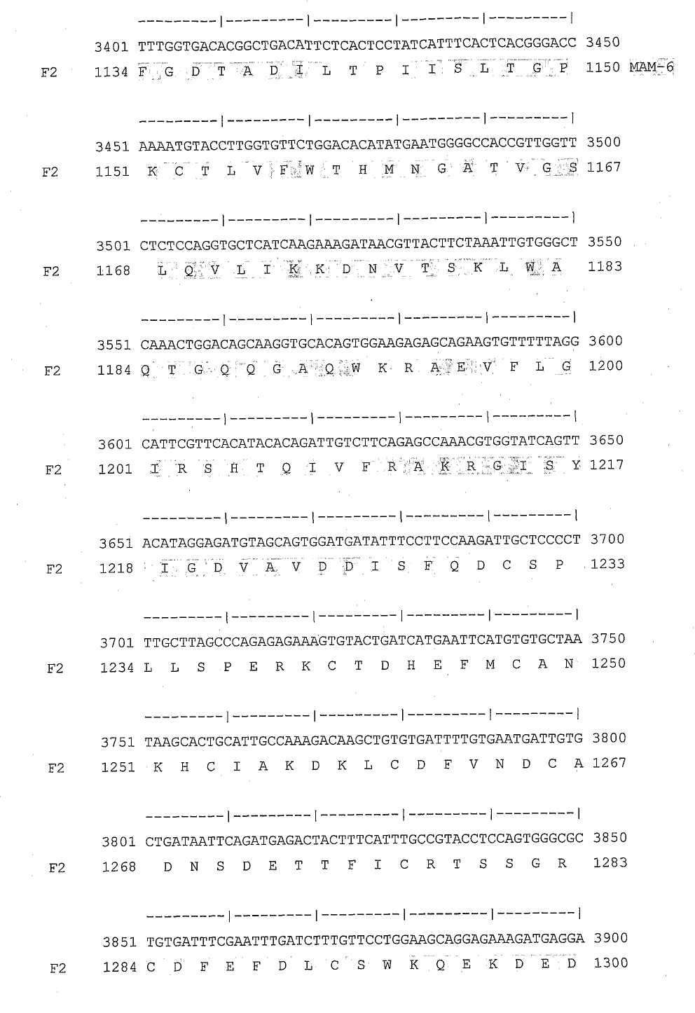

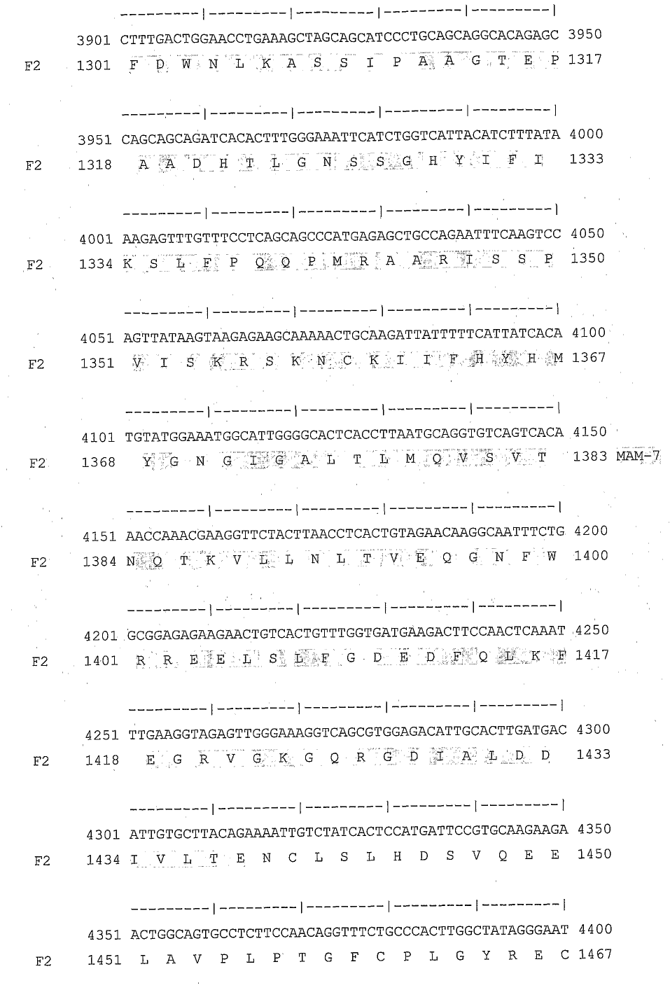

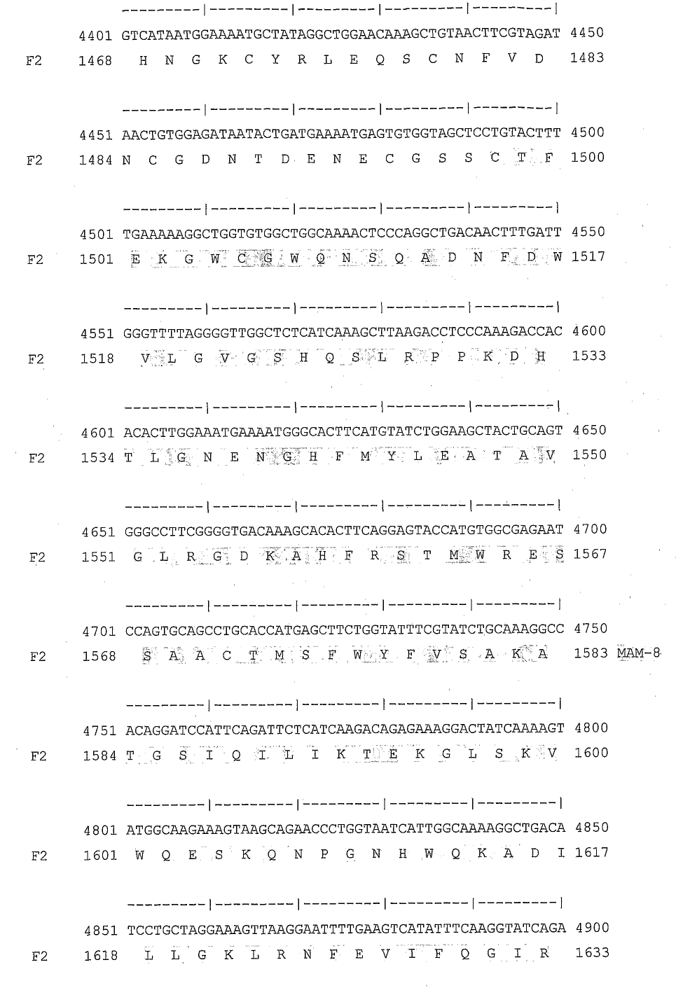

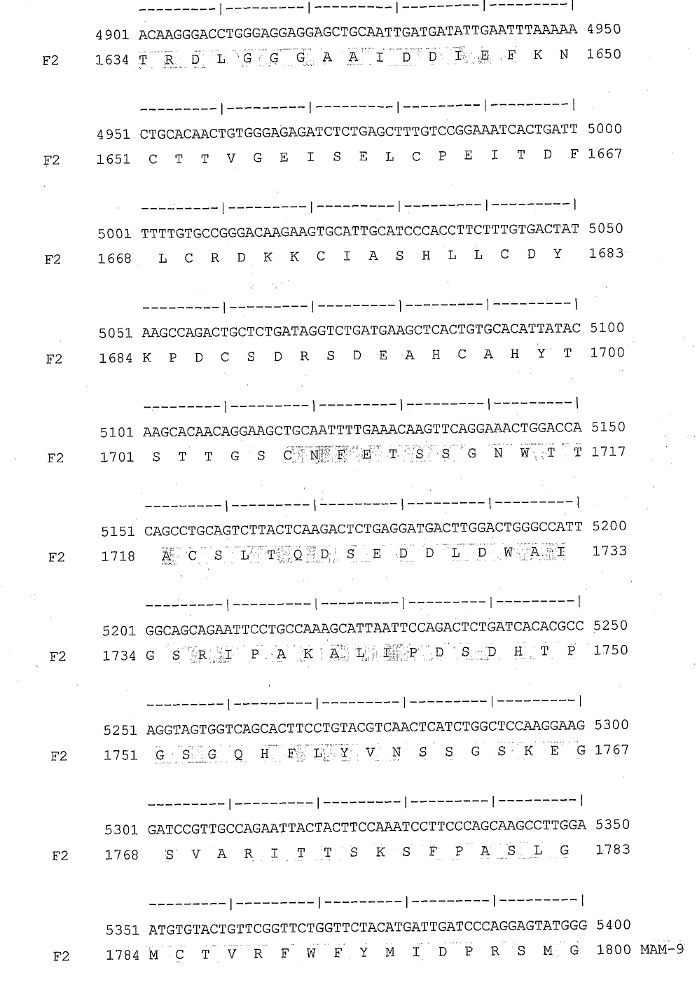

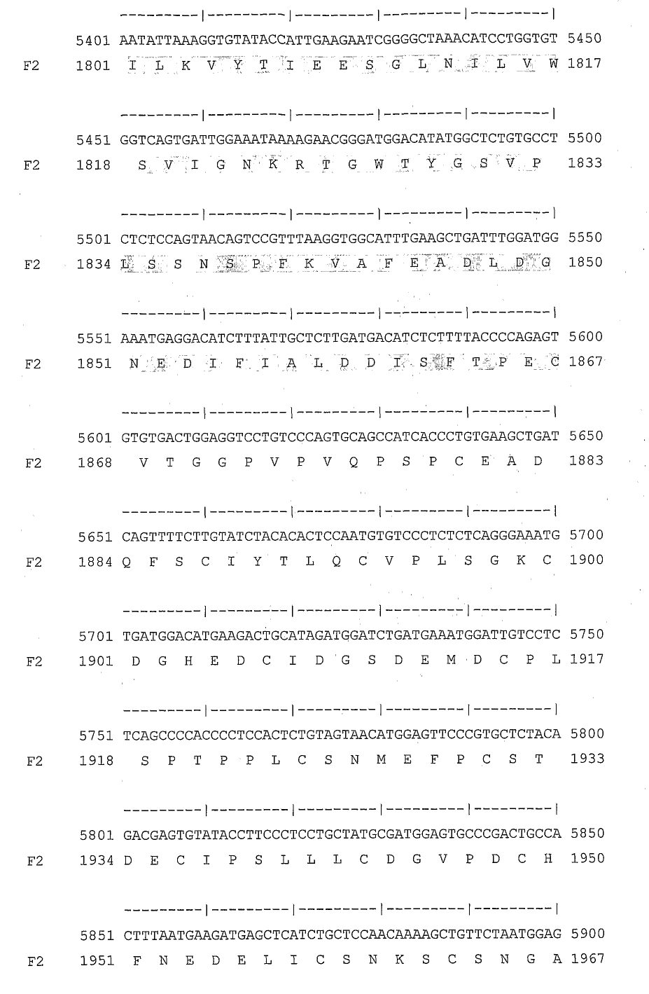

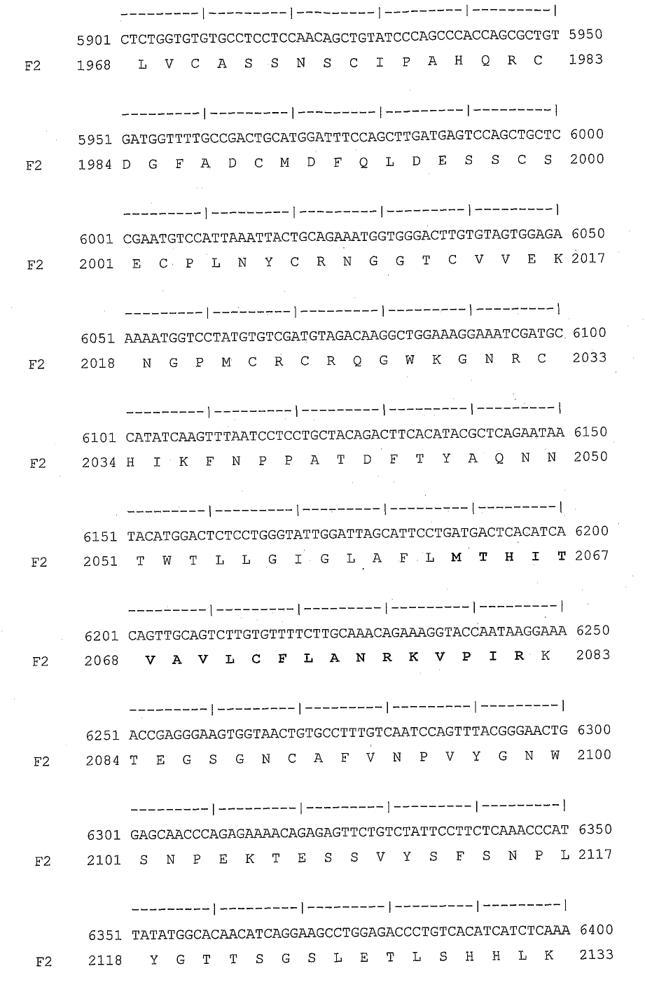

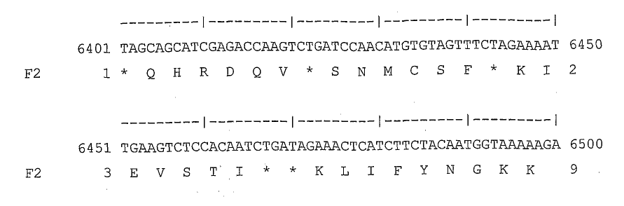

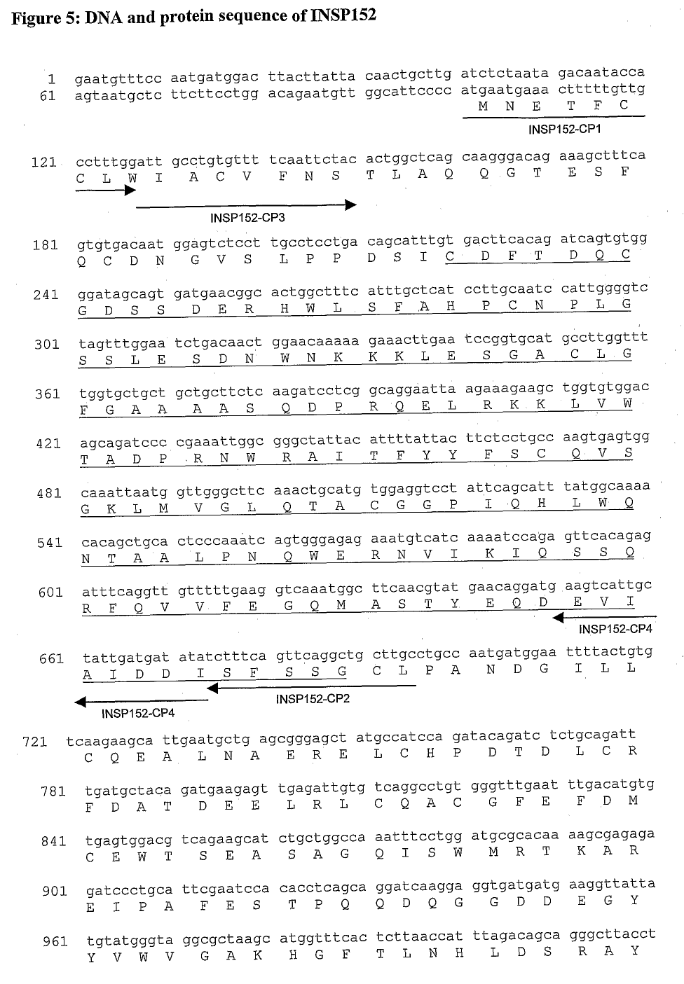

Gene-specific cloning primers INSP152-CP1 and INSP152-CP2 (Table 3, FIG. 5) were designed to amplify a cDNA fragment of 595 bp containing the entire INSP152 domain 1 sequence. The primer pair was used with the pools of human cDNA samples described above as PCR templates. This PCR1 was performed in a final volume of 50 μl containing 1× AmpliTaq™ buffer, 200 μM dNTPs, 50 pmoles of INSP152-CP1, 50 pmoles of INSP152-CP2, 2.5 units of AmpliTaq™ (Applied Biosystems) and approximately 10 ng of pool cDNA using an MJ Research DNA Engine, programmed as follows: 94° C., 2 min; 40 cycles of 94° C., 1 min, 60° C., 1 min, and 72° C., 1 min; followed by 1 cycle at 72° C. for 7 min and a holding cycle at 4° C.

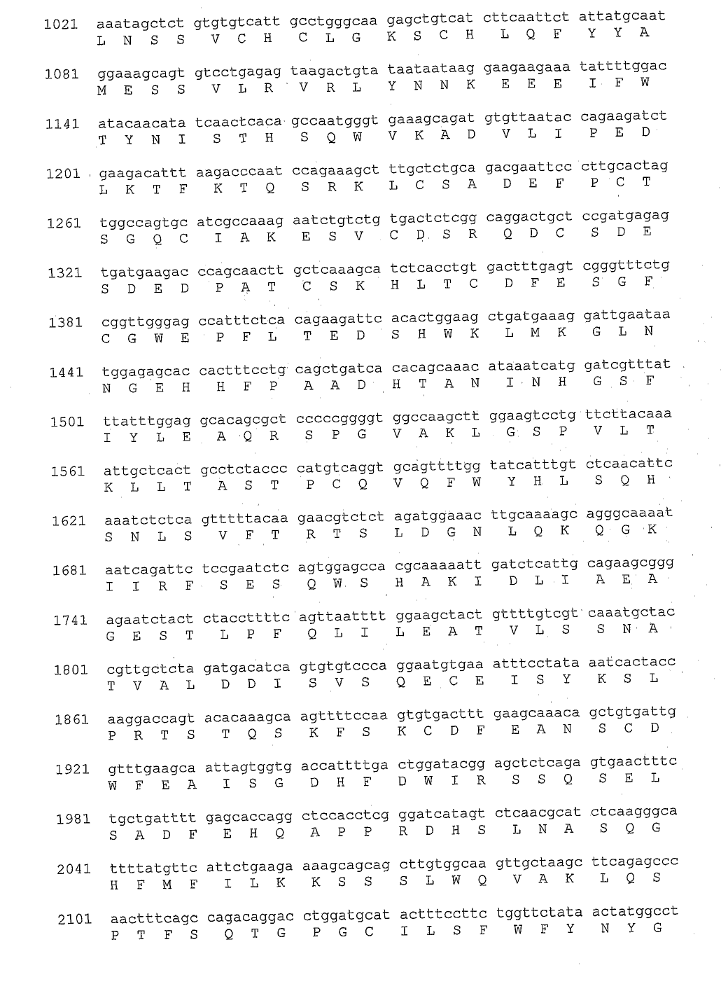

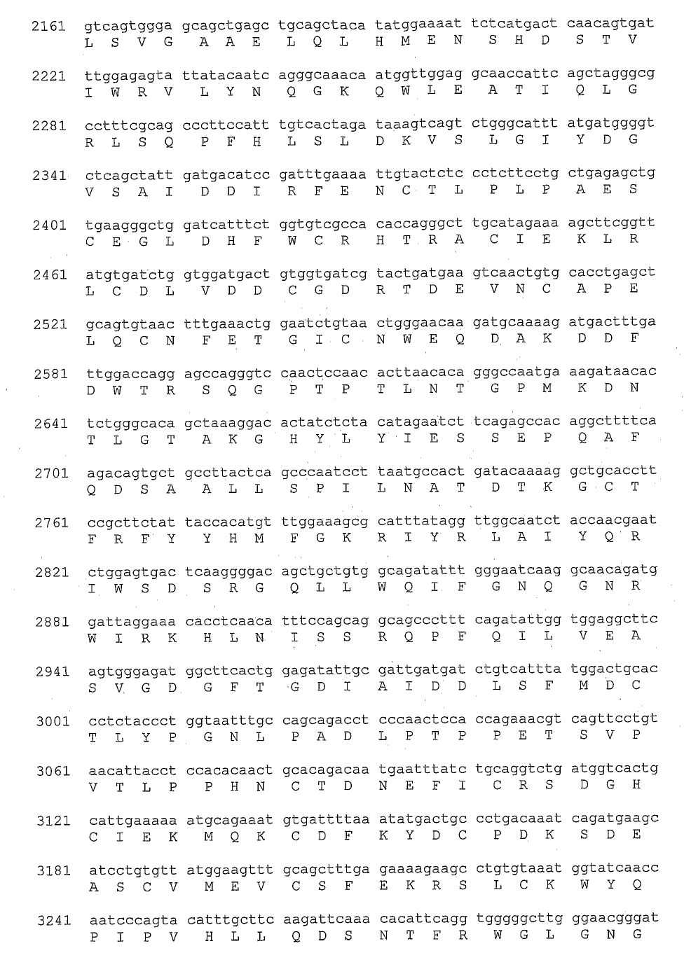

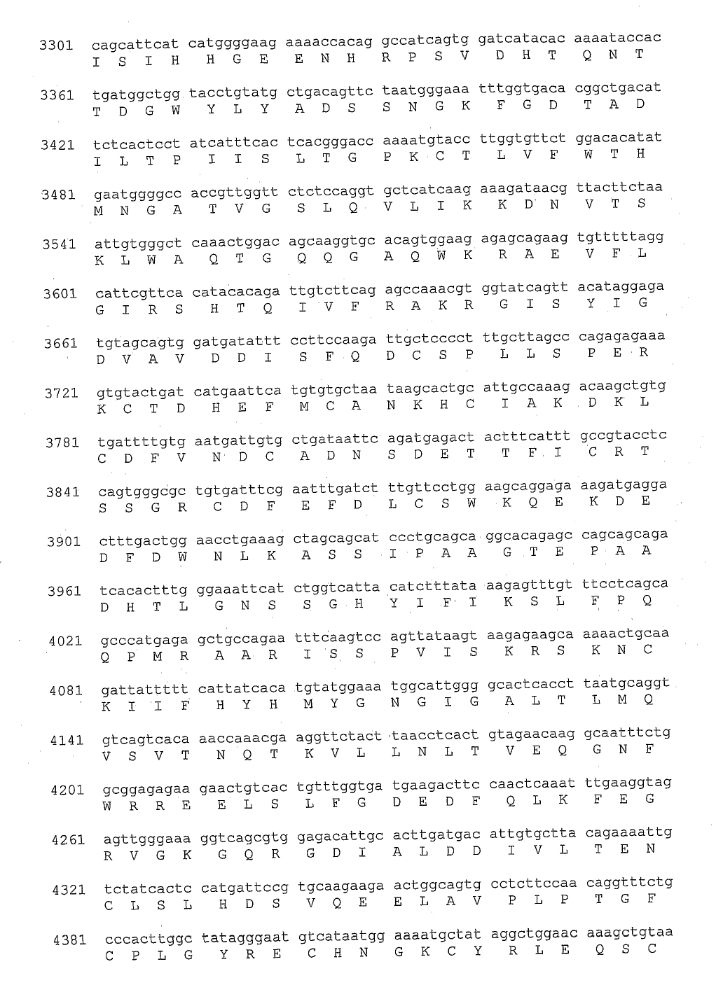

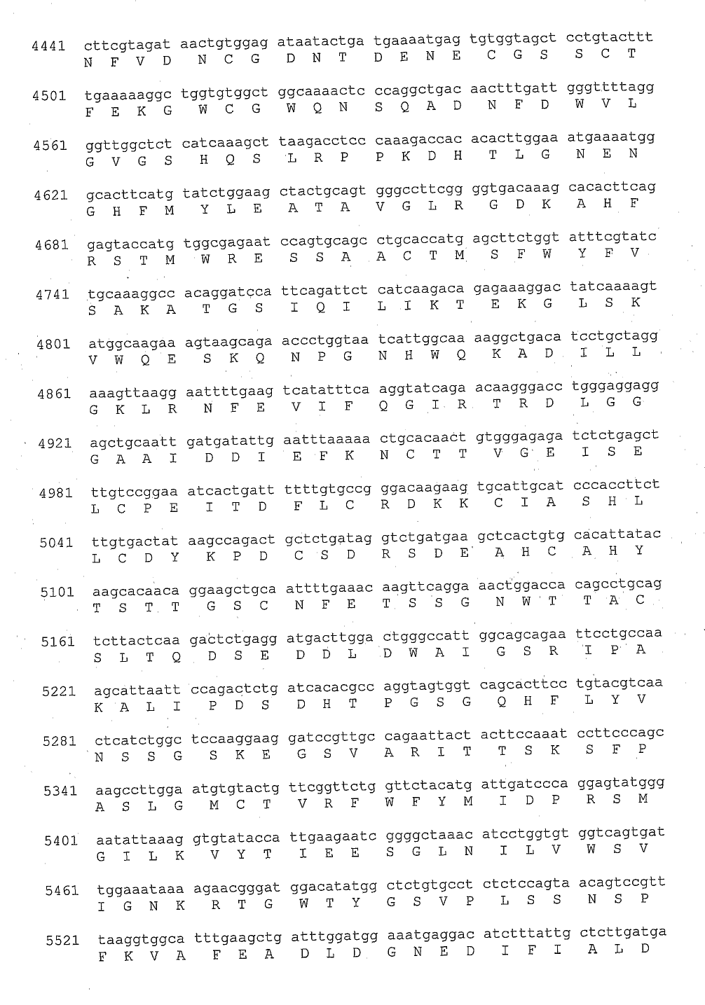

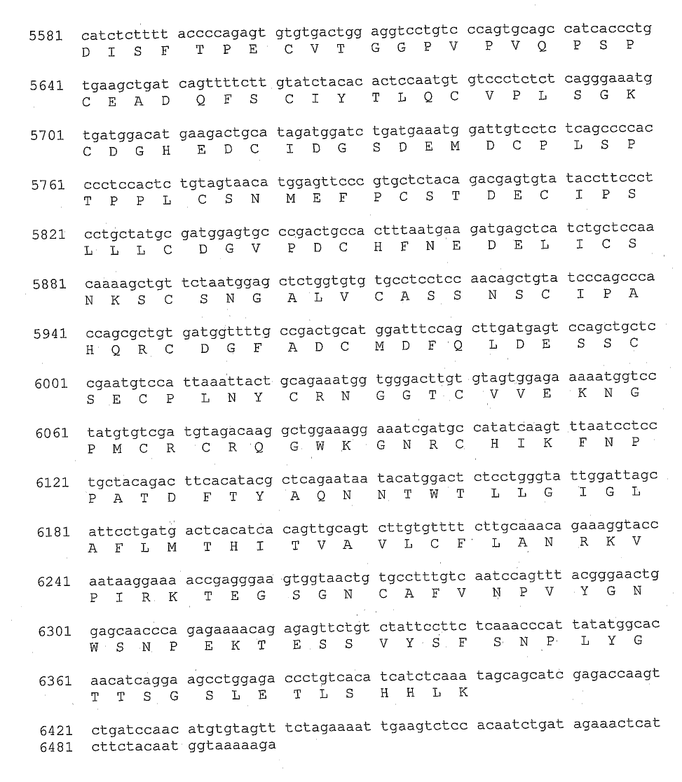

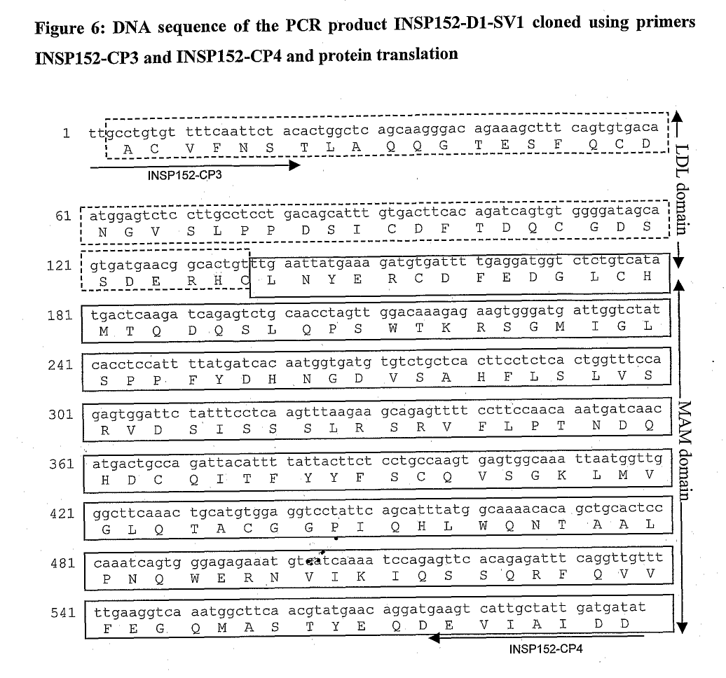

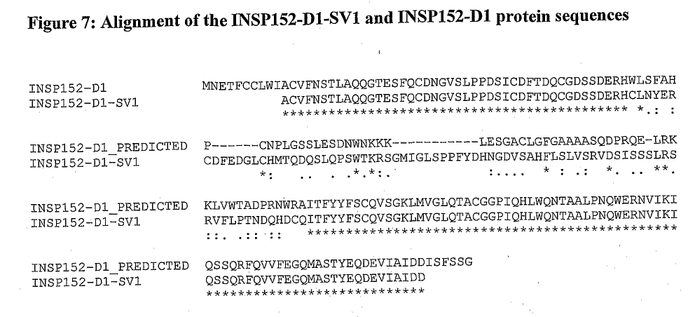

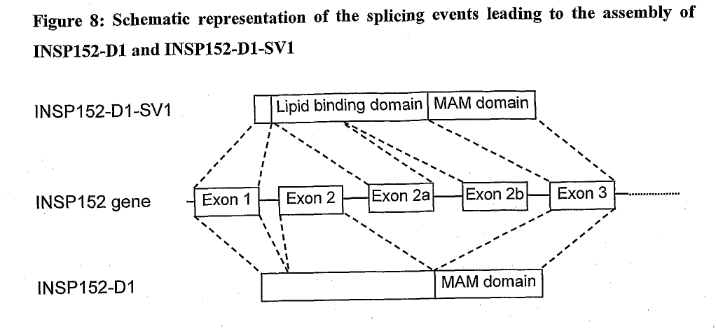

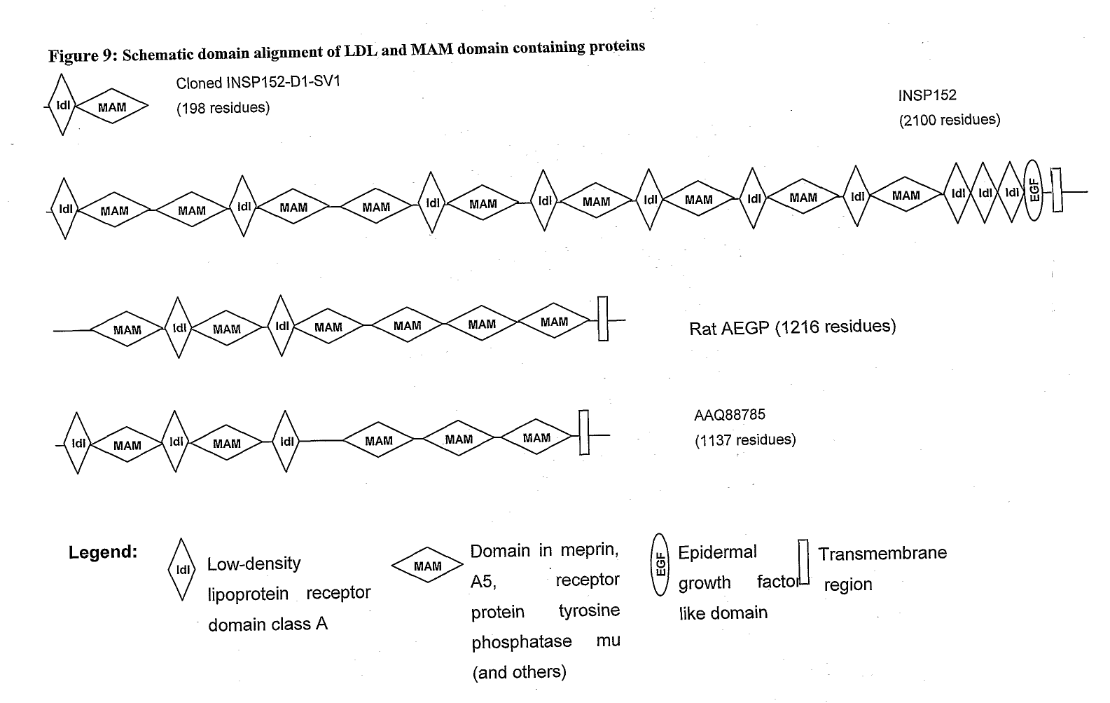

Each PCR1 product was then used as template for PCR2 using amplification primers INSP152-CP3 and INSP152-CP4 (Table 3, FIG. 5, FIG. 6, FIG. 7) designed to amplify a cDNA fragment of 546 bp within the INSP152-CP1/INSP152-CP2 product. PCR2 was performed in a final volume of 50 μl containing 1× AmpliTaq™ buffer, 200 μM dNTPs, 50 pmoles of INSP152-CP3, 50 pmoles of INSP152-CP4, 2.5 units of AmpliTaq™ (Applied Biosystems) and 1 μl of PCR1 product. Cycling was performed using an MJ Research DNA Engine, programmed as follows: 94° C., 2 min; 40 cycles of 94° C., 1 min, 57° C., 1 min, and 72° C., 1 min; followed by 1 cycle at 72° C. for 7 min and a holding cycle at 4° C.

30 μl of each PCR1 and PCR2 amplification product was visualized on a 0.8% agarose gel in 1×TAE buffer (Invitrogen). Products of the approximately the expected molecular weight were purified from the gel using either the Wizard PCR Preps DNA Purification System (Promega) and eluted in 50 μl of water, or the MinElute DNA Purification System (Qiagen) and eluted in 10 μl of EB buffer (10 mM Tris.Cl, pH 8.5). The purified products were subcloned directly.

### 4.4 Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 seconds. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

### 4.5 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Applied Biosystems) using an MJ Research DNA Engine. The cycling conditions were as follows: 94° C., 2 min; 30 cycles of 94° C., 30 sec, 48° C., 30 sec and 72° C. for 1 min. Samples were maintained at 4° C. (holding cycle) before further analysis.

PCR products were analyzed on 1% agarose gels in 1×TAE buffer. Colonies which gave PCR products of approximately the expected molecular weight (595 bp or 546 bp+105 bp due to the multiple cloning site (MCS)) were grown up overnight at 37° C. in 5 ml L-Broth (LB) containing ampicillin (100 μg/ml), with shaking at 220 rpm.

### 4.6 Plasmid DNA Preparation and Sequencing

Miniprep plasmid DNA was prepared from the 5 ml cultures using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Plasmid DNA (200-500 ng) was subjected to DNA sequencing with the T7 and T3 sequencing primers (Table 3) using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analyzed on an Applied Biosystems 3700 sequencer.

TABLE 3
INSP152-D1 cloning and sequencing primers

| | |
|---|---|
| Primer | Sequence (5′-3′) |

| | | |
|---|---|---|
| INSP152-CP1 | ATG AAT GAA ACT TTT TGT TGC CTT TGG | (SEQ ID NO: 30) |
| INSP152-CP2 | GCA AGC AGC CTG AAC TGA AAG A | (SEQ ID NO: 31) |
| INSP152-CP3 | ATT GCC TGT GTT TTC AAT TCT AC | (SEQ ID NO: 32) |
| INSP152-CP4 | TAT ATC ATC AAT AGC AAT GAC TTC | (SEQ ID NO: 33) |
| T7 | TAA TAC GAC TCA CTA TAG G | (SEQ ID NO: 34) |
| T3 | ATT AAC CCT CAC TAA AGG | (SEQ ID NO: 35) |

Underlined sequence = Kozak sequence
Bold = Stop codon
Italic sequence = His tag

### Example 5

### Expression and Purification of INSP152

Further experiments may now be performed to determine the tissue distribution and expression levels of the INSP152 polypeptides in vivo, on the basis of the nucleotide and amino acid sequence disclosed herein.

The presence of the transcripts for INSP152 may be investigated by PCR of cDNA from different human tissues. The INSP152 transcripts may be present at very low levels in the samples tested. Therefore, extreme care is needed in the design of experiments to establish the presence of a transcript in various human tissues as a small amount of genomic contamination in the RNA preparation will provide a false positive result. Thus, all RNA should be treated with DNAse prior to use for reverse transcription. In addition, for each tissue a control reaction may be set up in which reverse transcription was not undertaken (a -ve RT control).

For example, 1 μg of total RNA from each tissue may be used to generate cDNA using Multiscript reverse transcriptase (ABI) and random hexamer primers. For each tissue, a control reaction is set up in which all the constituents are added except the reverse transcriptase (-ve RT control). PCR reactions are set up for each tissue on the reverse transcribed RNA samples and the minus RT controls. INSP152-specific primers may readily be designed on the basis of the sequence information provided herein. The presence of a product of the correct molecular weight in the reverse transcribed sample together with the absence of a product in the minus RT control may be taken as evidence for the presence of a transcript in that tissue. Any suitable cDNA libraries may be used to screen for the INSP152 transcripts, not only those generated as described above.

The tissue distribution pattern of the INSP152 polypeptides will provide further useful information in relation to the function of those polypeptides.

In addition, further experiments may now be performed using expression vectors. Transfection of mammalian cell lines with such vectors may enable the high level expression of the INSP152 polypeptides and thus enable the continued investigation of the functional characteristics of the INSP152 polypeptides. The following material and methods are an example of those suitable in such experiments:

### Cell Culture

Human Embryonic Kidney 293 cells expressing the Epstein-Barr virus Nuclear Antigen (HE 93-EBNA, Invitrogen) are maintained in suspension in Ex-cell VPRO serum-free medium (seed stock, maintenance medium, JRH). Sixteen to 20 hours prior to transfection (Day-1), cells are seeded in 2× T225 flasks (50 ml per flask in DMEM/F12 (1:1) containing 2% FBS seeding medium (JRH) at a density of 2×10⁵ cells/ml). The next day (transfection day 0) transfection takes place using the JetPEITM reagent (2 μl/μg of plasmid DNA, PolyPlus-transfection). For each flask, plasmid DNA is co-transfected with GFP (fluorescent reporter gene) DNA. The transfection mix is then added to the 2× T225 flasks and incubated at 37° C. (5% CO₂) for 6 days. Confirmation of positive transfection may be carried out by qualitative fluorescence examination at day 1 and day 6 (Axiovert 10 Zeiss).

On day 6 (harvest day), supernatants from the two flasks are pooled and centrifuged (e.g. 4° C., 400 g) and placed into a pot bearing a unique identifier. One aliquot (500 μl) is kept for QC of the 6His-tagged protein (internal bioprocessing QC).

Scale-up batches may be produced by following the protocol called “PEI transfection of suspension cells”, referenced BP/PEI/HH/02/04, with PolyEthyleneImine from Polysciences as transfection agent.

### Purification Process

The culture medium sample containing the recombinant protein with a C-terminal 6His tag is diluted with cold buffer A (50 mM NaH₂PO₄; 600 mM NaCl; 8.7% (w/v) glycerol, pH 7.5). The sample is filtered then through a sterile filter (Millipore) and kept at 4° C. in a sterile square media bottle (Nalgene).

The purification is performed at 4° C. on the VISION workstation (Applied Biosystems) connected to an automatic sample loader (Labomatic). The purification procedure is composed of two sequential steps, metal affinity chromatography on a Poros 20 MC (Applied Biosystems) column charged with Ni ions (4.6×50 mm, 0.83 ml), followed by gel filtration on a Sephadex G-25 medium (Amersham Pharmacia) column (1.0×10 cm).

For the first chromatography step the metal affinity column is regenerated with 30 column volumes of EDTA solution (100 mM EDTA; 1M NaCl; pH 8.0), recharged with Ni ions through washing with 15 column volumes of a 100 mM NiSO₄ solution, washed with 10 column volumes of buffer A, followed by 7 column volumes of buffer B (50 mM NaH₂PO₄; 600 mM NaCl; 8.7% (w/v) glycerol, 400 mM; imidazole, pH 7.5), and finally equilibrated with 15 column volumes of buffer A containing 15 mM imidazole. The sample is transferred, by the Labomatic sample loader, into a 200 ml sample loop and subsequently charged onto the Ni metal affinity column at a flow rate of 10 ml/min. The column is washed with 12 column volumes of buffer A, followed by 28 column volumes of buffer A containing 20 mM imidazole. During the 20 mM imidazole wash loosely attached contaminating proteins are eluted from the column. The recombinant His-tagged protein is finally eluted with 10 column volumes of buffer B at a flow rate of 2 ml/min, and the eluted protein is collected.

For the second chromatography step, the Sephadex G-25 gel-filtration column is regenerated with 2 ml of buffer D (1.137M NaCl; 2.7 mM KCl; 1.5 mM KH₂PO₄; 8 mM Na₂HPO₄; pH 7.2), and subsequently equilibrated with 4 column volumes of buffer C (137 mM NaCl; 2.7 mM KCl; 1.5 mM KH₂PO₄; 8 mM Na₂HPO₄; 20% (w/v) glycerol; pH 7.4). The peak fraction eluted from the Ni-column is automatically loaded onto the Sephadex G-25 column through the integrated sample loader on the VISION and the protein is eluted with buffer C at a flow rate of 2 ml/min. The fraction was filtered through a sterile centrifugation filter (Millipore), frozen and stored at −80° C. An aliquot of the sample is analyzed on SDS-PAGE (4-12% NuPAGE gel; Novex) Western blot with anti-H is antibodies. The NuPAGE gel may be stained in a 0.1% Coomassie blue R250 staining solution (30% methanol, 10% acetic acid) at room temperature for 1 h and subsequently destained in 20% methanol, 7.5% acetic acid until the background is clear and the protein bands clearly visible.

Following the electrophoresis the proteins are electrotransferred from the gel to a nitrocellulose membrane. The membrane is blocked with 5% milk powder in buffer E (137 mM NaCl; 2.7 mM KCl; 1.5 mM KH₂PO₄; 8 mM Na₂HPO₄; 0.1% Tween 20, pH 7.4) for 1 h at room temperature, and subsequently incubated with a mixture of 2 rabbit polyclonal anti-His antibodies (G-18 and H-15, 0.2 μg/ml each; Santa Cruz) in 2.5% milk powder in buffer E overnight at 4° C. After a further 1 hour incubation at room temperature, the membrane is washed with buffer E (3×10 min), and then incubated with a secondary HRP-conjugated anti-rabbit antibody (DAKO, HRP 0399) diluted 1/3000 in buffer E containing 2.5% milk powder for 2 hours at room temperature. After washing with buffer E (3×10 minutes), the membrane is developed with the ECL kit (Amersham Pharmacia) for 1 min. The membrane is subsequently exposed to a Hyperfilm (Amersham Pharmacia), the film developed and the western blot image visually analysed.

For samples that showed detectable protein bands by Coomassie staining, the protein concentration may be determined using the BCA protein assay kit (Pierce) with bovine serum albumin as standard.

Furthermore, overexpression or knock-down of the expression of the polypeptides in cell lines may be used to determine the effect on transcriptional activation of the host cell genome. Dimerisation partners, co-activators and co-repressors of the INSP152 polypeptides may be identified by immunoprecipitation combined with Western blotting and immunoprecipitation combined with mass spectroscopy.

### Example 6

### Analysis of INSP152 Gene Expression Levels by TaqMan Analysis

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, and dTTP, and DEPC-treated water in a volume of 10 μl. The mixture was incubated at 65° C. for 5 min then chilled on ice for 1 min. The following 10 μl cDNA synthesis mix was prepared in a separate tube: 21 μl 10×RT buffer, 4 μl 25 mM MgCl₂, 2 μl 0.1M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700).

PCR primers for human INSP152 and the housekeeping control gene glyceraldehyde 3-phosphate dehydrogenase (GAPDH) were designed using the Primer Express software (PE Biosystems). The PCR primers for INSP152 were designed with the forward primer in exon 4 and the reverse primer in exon 5. These primers would not distinguish between INSP152 from the prediction and the cloned INSP152-SV1-D1. The sequences of the primers are shown in Table 4. The specificity and the optimal primer concentration to use for the TaqMan analysis were determined by testing the INSP152 gene-specific primers on a series of dilutions of plasmid pEAK12d-PAC_INSP152-D1-SV1-6HIS-V1. Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

TABLE 4
TaqMan PCR primer sequences

| | |
|---|---|
| Primer | Sequence (5′-3′) |

| | |
|---|---|
| h-INSP152-D1-SV1-519F | GGAGAGAAATGTCATCAAAATCCA |
| | (SEQ ID NO: 36) |
| h-INSP152-D1-SV1-584R | GCCATTTGACCTTCAAAAACAAC |
| | (SEQ ID NO: 37) |
| hGAPDH-F | CCACCCATGGCAAATTCC |
| | (SEQ ID NO: 38) |
| hGAPDH-R | GATGGGATTTCCATTGATGACA |
| | (SEQ ID NO: 39) |
| Intron-hGAPDH-F | CCTAGTCCCAGGGCTTTGATT |
| | (SEQ ID NO: 40) |
| Intron-hGAPDH-R | CTGTGCTCCCACTCCTGATTT |
| | (SEQ ID NO: 41) |

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the INSP152 gene in each cDNA sample was expressed as a difference in Ct value, i.e. Delta (δ) Ct=Ct (GAPDH)−Ct (INSP152). Results for each sample were then expressed as a fold difference in the number of cycles required for detectable INSP152 gene expression relative to that for GAPDH, according to the formula Fold Difference=2^{(-δCt)}. Finally, the expression level of the INSP152 gene in each cDNA sample was shown relative to the GAPDH gene expression level, where GAPDH expression level=100%, by dividing 100 by the Fold Difference for INSP152.

### Results

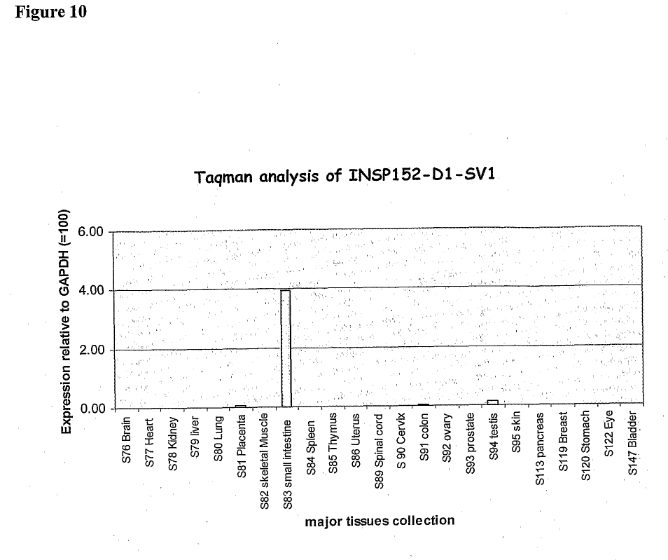

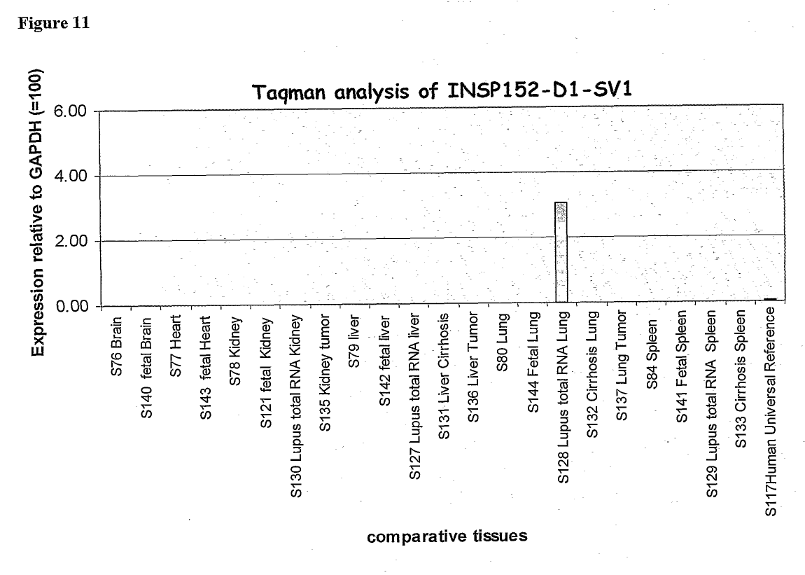

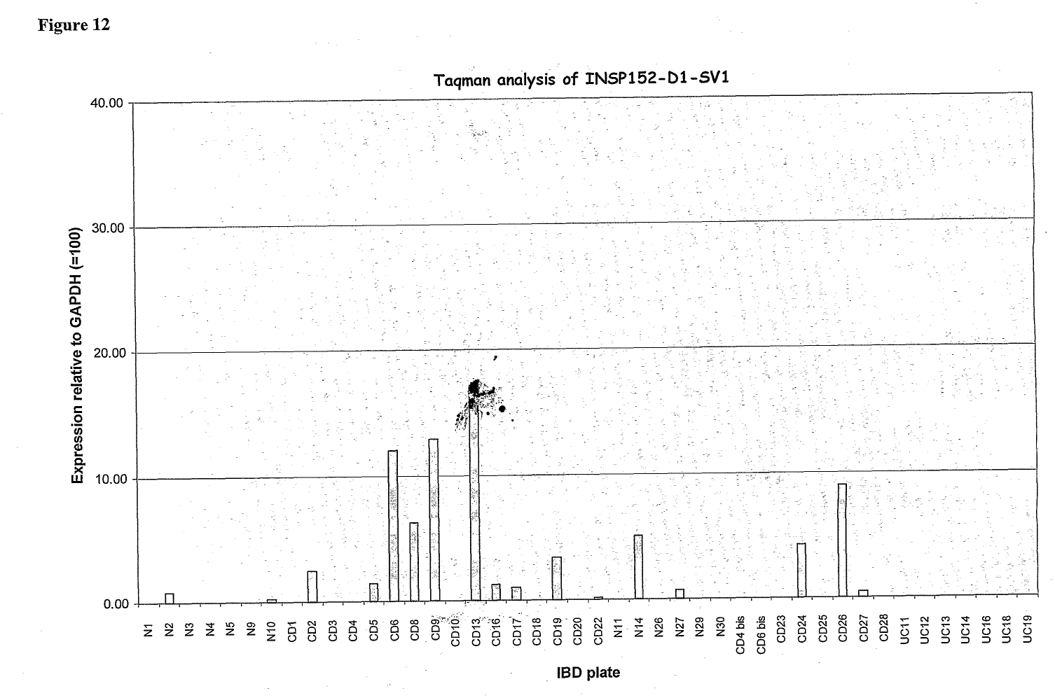

INSP152 primers were tested on a panel of normal and diseased human tissue samples containing 23 major organs; 21 secretory and immune tissues; 43 primary cells and cell lines of non-leukocytic, immune and CNS origin and colon or ileum biopsies from Crohn's disease (CD) (22 samples), ulcerative colitis (UC) (7 samples) and normal colon or ileum (N) resected from patients undergoing surgery for bowel cancer (13 samples). Results are shown in tables 5, 6 and 7 and represented graphically in FIGS. 10, 11 and 12. INSP152 was below the limit of detection in all samples tested except in small intestine (3.94% of GAPDH) (FIG. 10), a single lupus lung sample (3.06% of GAPDH) (FIG. 11) and in 12/22 Crohn's disease biopsy samples (FIG. 12). The 12 positive CD samples were all ileal biopsies.

### CONCLUSION

Expression results show unexpected restricted expression of INSP152 in small intestine (table 5 and FIG. 10), in a single lupus sample (table 6 and FIG. 11), and in 12/22 Crohn's disease biopsy samples (table 7 and FIG. 12). Except for one normal sample (5.09% of GADPH), all normal colon or ileum biopsies are below 0.8% of GADPH.

This specific pattern of expression leads to the conclusion of the involvement of INSP152, preferably INSP152-D1-SV1, in diseases of the small intestine, preferably Crohn's disease, small bowel disease, inflammatory bowel disease, lupus, celiac disease, Whipple's disease, neoplasias and diarrhea. These surprising properties characterizing the polynucleotides or the corresponding polypeptides of the present invention make them particularly suitable for the preparation of a drug or pharmaceutical composition. The polynucleotides or the corresponding polypeptides of the present invention therefore display the unexpected finding of a restricted expression in specific tissues.

TABLE 5
Expression of INSP152 in major human tissues as measured by RT-PCR
(TaqMan).

| | | | | | Relative |
|---|---|---|---|---|---|
| | | | | | to |
| major tissues | | Ct hINSP152 | | Fold | GAPDH |
| collection | Ct hGAPDH | D1-SV1 | delta ct | difference | (=100) |
| S76 Brain | 20.79 | 37.15 | −16.36 | 84110.60 | 0.00 |
| S77 Heart | 19.96 | 40.00 | −20.05 | 1081798.22 | 0.00 |
| S78 Kidney | 18.58 | 40.00 | −21.43 | 2815578.61 | 0.00 |
| S79 liver | 22.43 | 40.00 | −17.58 | 195255.00 | 0.00 |
| S80 Lung | 20.91 | 40.00 | −19.10 | 559973.88 | 0.00 |
| S81 Placenta | 20.26 | 30.50 | −10.24 | 1205.15 | 0.08 |
| S82 skeletal Muscle | 15.87 | 40.00 | −24.14 | 18422940.32 | 0.00 |
| S83 small intestine | 20.20 | 24.87 | −4.67 | 25.37 | 3.94 |
| S84 Spleen | 20.32 | 37.83 | −17.52 | 187301.12 | 0.00 |
| S85 Thymus | 19.22 | 40.00 | −20.78 | 1800542.22 | 0.00 |
| S86 Uterus | 20.23 | 34.72 | −14.49 | 23010.42 | 0.00 |
| S89 Spinal cord | 19.06 | 40.00 | −20.95 | 2018706.86 | 0.00 |
| S90 Cervix | 22.12 | 40.00 | −17.88 | 241221.67 | 0.00 |
| S91 colon | 20.15 | 31.83 | −11.68 | 3281.18 | 0.03 |
| S92 ovary | 21.91 | 37.07 | −15.17 | 36738.37 | 0.00 |
| S93 prostate | 19.02 | 40.00 | −20.98 | 2068279.89 | 0.00 |
| S94 testis | 20.21 | 29.71 | −9.51 | 726.59 | 0.14 |
| S95 skin | 23.47 | 40.00 | −16.54 | 94957.87 | 0.00 |
| S113 pancreas | 22.07 | 38.90 | −16.83 | 116502.39 | 0.00 |
| S119 Breast | 20.05 | 38.51 | −18.46 | 359342.49 | 0.00 |
| S120 Stomach | 20.11 | 34.39 | −14.29 | 19962.43 | 0.01 |
| S122 Eye | 21.53 | 40.00 | −18.48 | 364358.72 | 0.00 |
| S147 Bladder | 19.69 | 40.00 | −20.31 | 1299927.74 | 0.00 |

TABLE 6
Expression of INSP152 in comparative human tissues as measured by RT-PCR
(TaqMan).

| | | | | | Relative |
|---|---|---|---|---|---|
| | | Ct | | | to |
| | Ct | hINSP152- | | Fold | GAPDH |
| Comparative tissues | hGAPDH | D1-SV1 | delta ct | difference | (=100) |
| S76 Brain | 20.36 | 40.00 | −19.64 | 817013.28 | 0.00 |
| S140 fetal Brain | 18.46 | 36.67 | −18.21 | 303218.87 | 0.00 |
| S77 Heart | 20.57 | 40.00 | −19.43 | 706338.40 | 0.00 |
| S143 fetal Heart | 17.82 | 37.55 | −19.73 | 866595.97 | 0.00 |
| S78 Kidney | 20.11 | 40.00 | −19.89 | 971598.00 | 0.00 |
| S121 fetal Kidney | 18.70 | 32.05 | −13.36 | 10477.45 | 0.01 |
| S130 Lupus total RNA Kidney | 21.48 | 40.00 | −18.52 | 375902.76 | 0.00 |
| S135 Kidney tumor | 18.86 | 39.13 | −20.27 | 1264381.15 | 0.01 |
| S79 liver | 22.42 | 40.00 | −17.59 | 196613.11 | 0.00 |
| S142 fetal liver | 18.89 | 40.00 | −21.12 | 2271163.28 | 0.00 |
| S127 Lupus total RNA liver | 23.45 | 36.31 | −12.87 | 7460.21 | 0.01 |
| S131 Liver Cirrhosis | 17.41 | 35.91 | −18.51 | 372014.67 | 0.00 |
| S136 Liver Tumor | 20.66 | 40.00 | −19.34 | 663620.99 | 0.00 |
| S80 Lung | 22.39 | 39.07 | −16.69 | 105362.36 | 0.00 |
| S144 Fetal Lung | 17.42 | 40.00 | −22.59 | 6291619.53 | 0.00 |
| S128 Lupus total RNA Lung | 19.91 | 24.94 | −5.03 | 32.67 | 3.06 |
| S132 Cirrhosis Lung | 16.56 | 40.00 | −23.45 | 11419530.49 | 0.00 |
| S137 Lung Tumor | 21.42 | 40.00 | −18.59 | 393226.22 | 0.00 |
| S84 Spleen | 20.84 | 40.00 | −19.17 | 587813.92 | 0.00 |
| S141 Fetal Spleen | 20.14 | 40.00 | −19.86 | 951602.81 | 0.00 |
| S129 Lupus total RNA Spleen | 21.04 | 40.00 | −18.96 | 509951.32 | 0.00 |
| S133 Cirrhosis Spleen | 20.99 | 40.00 | −19.01 | 527934.71 | 0.00 |
| S117Human Universal Reference | 15.67 | 27.84 | −12.17 | 4592.30 | 0.02 |

TABLE 7
Expression of INSP152 in diseased colon and ileum as measured
by RT-PCR (TaqMan).

| | | | | | Relative |
|---|---|---|---|---|---|
| | | | | | to |
| | Ct | Ct hINSP152- | | Fold | GAPDH |
| IBD plate | hGAPDH | D1-SV1 | delta ct | difference | (=100) |
| N1 | 23.82 | 39.87 | −16.05 | 67612.39 | 0.00 |
| N2 | 22.09 | 29.13 | −7.04 | 131.60 | 0.76 |
| N3 | 23.66 | 40.00 | −16.35 | 83240.61 | 0.00 |
| N4 | 20.63 | 36.43 | −15.80 | 56855.02 | 0.00 |
| N5 | 20.44 | 40.00 | −19.56 | 772941.66 | 0.00 |
| N9 | 22.46 | 40.00 | −17.55 | 191236.71 | 0.00 |
| N10 | 21.52 | 30.10 | −8.59 | 384.01 | 0.26 |
| CD1 | 23.41 | 40.00 | −16.59 | 98647.35 | 0.00 |
| CD2 | 21.04 | 26.41 | −5.37 | 41.21 | 2.43 |
| CD3 | 18.28 | 29.81 | −11.53 | 2957.17 | 0.03 |
| CD4 | 20.92 | 40.00 | −19.09 | 556105.85 | 0.00 |
| CD5 | 25.31 | 31.47 | −6.16 | 71.26 | 1.40 |
| CD6 | 22.54 | 25.59 | −3.05 | 8.28 | 12.07 |
| CD8 | 22.06 | 26.06 | −4.00 | 15.94 | 6.27 |
| CD9 | 22.24 | 25.19 | −2.95 | 7.73 | 12.94 |
| CD10 | 22.35 | 40.00 | −17.65 | 205674.01 | 0.00 |
| CD13 | 22.52 | 25.06 | −2.55 | 5.84 | 17.13 |
| CD16 | 23.40 | 29.68 | −6.28 | 77.71 | 1.29 |
| CD17 | 24.78 | 31.42 | −6.64 | 99.73 | 1.00 |
| CD18 | 25.89 | 39.75 | −13.87 | 14920.41 | 0.01 |
| CD19 | 21.31 | 26.17 | −4.87 | 29.14 | 3.43 |
| CD20 | 21.87 | 40.00 | −18.13 | 286862.53 | 0.00 |
| CD22 | 21.26 | 30.82 | −9.57 | 757.45 | 0.13 |
| N11 | 23.67 | 39.15 | −15.48 | 45544.84 | 0.00 |
| N14 | 20.86 | 25.15 | −4.30 | 19.63 | 5.09 |
| N26 | 20.97 | 40.00 | −19.03 | 535304.41 | 0.00 |
| N27 | 21.74 | 28.92 | −7.19 | 145.51 | 0.69 |
| N29 | 23.27 | 40.00 | −16.73 | 108700.57 | 0.00 |
| N30 | 21.10 | 38.97 | −17.87 | 239555.43 | 0.00 |
| CD4 bis | 21.68 | 38.78 | −17.11 | 140967.20 | 0.00 |
| CD6 bis | 20.71 | 36.08 | −15.37 | 42347.82 | 0.00 |
| CD23 | 24.62 | 40.00 | −15.38 | 42642.37 | 0.00 |
| CD24 | 21.86 | 26.41 | −4.56 | 23.51 | 4.25 |
| CD25 | 21.92 | 38.66 | −16.75 | 109836.65 | 0.00 |
| CD26 | 21.96 | 25.45 | −3.49 | 11.20 | 8.93 |
| CD27 | 22.69 | 30.48 | −7.80 | 222.09 | 0.45 |
| CD28 | 22.11 | 35.28 | −13.17 | 9184.59 | 0.01 |
| UC11 | 23.05 | 40.00 | −16.96 | 127046.75 | 0.00 |
| UC12 | 21.14 | 40.00 | −18.87 | 477453.27 | 0.00 |
| UC13 | 21.16 | 38.65 | −17.50 | 184722.49 | 0.00 |
| UC14 | 21.93 | 40.00 | −18.08 | 276132.28 | 0.00 |
| UC16 | 22.86 | 39.94 | −17.08 | 138066.14 | 0.00 |
| UC18 | 23.05 | 36.15 | −13.10 | 8779.97 | 0.01 |
| UC19 | 23.10 | 36.19 | −13.09 | 8719.32 | 0.01 |

### Example 7

### Human Mixed Lymphocyte Reaction (hMLR): Proliferation and Cytokine Secretion

This cell-based assay addresses antigen-specific T cell and antigen presenting cell functions upon stimulation by PBMC from another donor (alloreactivity). These are crucial cellular responses in inflammatory and autoimmune diseases. This assay allows the identification of compounds that have an effect on lymphocyte proliferation and/or cytokine secretion.

By analysing the cytokine secretion in this assay, it allows us to reveal if a compound is able to drive the polarization of the T lymphocytes to a TH-1 or TH-2 phenotype.

As an example, Multiple Sclerosis is associated with Th-1 cytokine profile (e.g.: γ-IFN, IL-2, TNF-α). With this assay it would be possible to identify compounds, which could inhibit TH-1 pro-inflammatory cytokine production or induced TH-2 anti-inflammatory cytokines (e.g.: IL-4, IL-5, IL-10).

hMLR Proliferation Assay for Dose-Response Determination-Experimental Design

10⁵ Stimulated cells (S), 2×10⁵ Responder cells (R) and the diluted protein of interest, i.e. INSP152-D1-SV1or CTLA4-Ig as a positive control, (starting dilution to have 2% glycerol maximum then seven ⅓ dilutions) were added, in complete medium (DMEM F12, GIBCO, Invitrogen Corporation, supplemented with 10% FCS, CANSERA inactived 50′ at 56° C., 10% L-Glutamine GIBCO, Invitrogen Corporation, 10% β-mercaptoethanol, FLUKA and 10% Penicillin/Streptomycin, GIBCO, Invitrogen Corporation) to a 96 well plate, round bottom (COSTAR). Four controls were performed. The positive control consisted in the S and R cells mix, the inhibition control in the S and R cells mix with Cyclosporin A 10⁻⁶ M (ALEXIS) and the last two controls in the S cells alone and R cells alone (negative control).

The cells were put in culture (37° C.) for four days. At day 3 μCi/well of ³H-Thymidine (AMERSHAM) was added. At day 4 the plates were harvested (OptiPhase “HiSafe”2, liquid scintillation cocktail, Perkin Elmer life sciences 1295-004 and Betaplate ±96-well harvester, Wallac) and the β-radiations counted (1205 Betaplate® liquid scintillation counter, Wallac). As an alternative, 1 μCi/well of ³H-Thymidine (AMERSHAM) was added at day 4 for 6 hours.

Plate format

| | | | | | | | 1/ | | |
|---|---|---|---|---|---|---|---|---|---|
| S | 1/10 | 1/30 | 1/90 | 1/270 | 1/810 | 1/2430 | 7290 | 1/21870 | R |
| S | | | | | | | | | R |
| S + R | | | | | | | | | R |

| | |
|---|---|
| S + R | S + R + |
| | CycloA |
| S + R | S + R + |
| | CycloA |
| S + R | S + R + |
| | CycloA |

### CONCLUSION

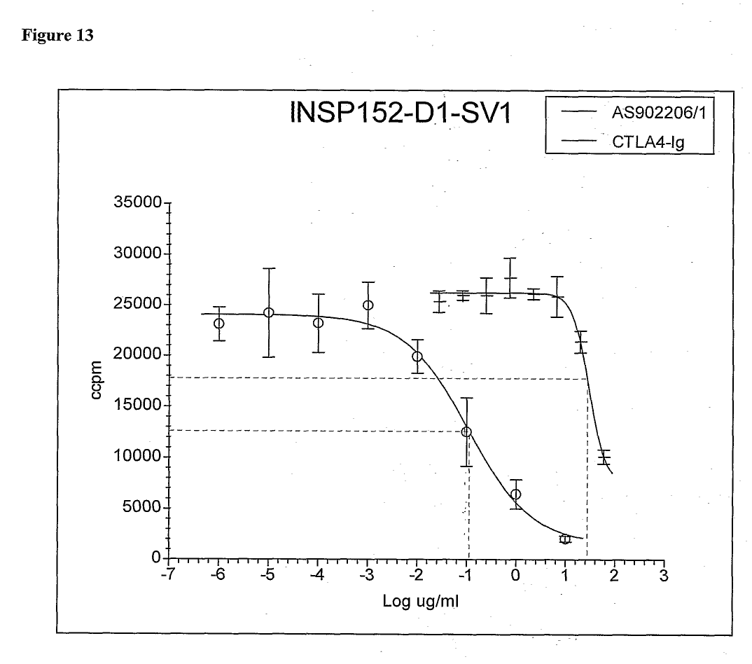

Results (FIG. 13) surprisingly show that INSP152-D1-SV1 is a potent inhibitor of lymphocytes, preferably of T cells. As such, INSP152-D1-SV1 displays similar functional activity as CTLA4-Ig. These surprising properties characterizing the polynucleotides or the corresponding polypeptides of the present invention make them particularly suitable for the preparation of a drug or pharmaceutical composition.

CTLA4-Ig is a soluble chimeric protein consisting of the extracellular domain of human CTLA4 and a fragment of the Fc portion of human IgG1. It binds to B7-1 and to B7-2 molecules on antigen-presenting cells (APCs) and thereby blocks the CD28-mediated costimulatory signal for T-cell activation and suppresses as well B cell response.

Biologic activity of CTLA4-Ig has been demonstrated in a variety of animal models of transplantation and autoimmunity. CTLA4-Ig has also been implicated or used in many diseases (see for example Davidson et al. Block and tackle: CTLA4Ig takes on lupus. Lupus. 2005; 14(3):197-203.). In addition, CTLA4-Ig has been developed as a drug for the treatment of various conditions. For example, Bristol-Myers Squibb developed abatacept for the treatment of multiple sclerosis, psoriasis, rheumatoid arthritis, systemic lupus erythematosus, transplant rejection, graft versus host disease. Repligen Corp developed RG-1046 for the treatment of thrombocytopenic purpura, multiple sclerosis, rheumatoid arthritis and graft versus host disease.

As such, polypeptides of the present invention, preferably INSP152-D1-SV1, are useful for the treatment of CTLA4-Ig related diseases. INSP152-D1-SV1 is useful on its own, as a component of fusion proteins such as Fc fusion, and/or in combination with another agent.

We claim:

1-56. (canceled)

57. A composition of matter comprising:
a) an isolated polypeptide selected from the group consisting of:
1) an amino acid sequence comprising SEQ ID NO:1;
2) an amino acid sequence comprising SEQ ID NO:2;
3) a fragment of said amino acid sequence which functions as a secreted protein or has an antigenic determinant in common with the polypeptide of 1);
4) a functional equivalent of 1) or 2);
5) an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18;
6) a fragment of said amino acid sequence of 5) which functions as a secreted protein or has an antigenic determinant in common with the polypeptide of 5);
7) a functional equivalent of 5);
8) a functional equivalent of 4) or 7), characterized in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; and is a LDL and/or MAM domain containing polypeptide;
9) the functional equivalent of 4), 7), or 8), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
10) the functional equivalent of 4), 7), or 8), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
11) a functional equivalent of 4), 7), 8), 9), or 10), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
12) the fragment of 3) or 6), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
13) the fragment of 3) or 6), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
14) the fragment of 3), 12), or 13), wherein the fragment has an antigenic determinant in common with the polypeptide of 1) or 2), or with the polypeptide of 5) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
15) the polypeptide of any one of 1) to 7), further comprising a histidine tag;
16) the polypeptide of any one of 1) to 7) or 15), wherein the polypeptide comprises a signal peptide;
17) the polypeptide of 16), whose sequence is recited in SEQ ID NO:20, SEQ ID NO:22, SEQ ID NO:24, SEQ ID NO:26, and/or SEQ ID NO:28;
18) a fusion polypeptide comprising a polypeptide of any one of 1) to 17); and
19) the fusion polypeptide of 18), comprising SEQ ID NO:4, SEQ ID NO:6, or SEQ ID NO:22; or
b) a purified nucleic acid molecule:
1) comprising a nucleic acid sequence encoding a polypeptide of any one of a1) to a19); or
2) comprising a nucleic acid sequence selected from the group consisting of SEQ ID NO:1, SEQ ID NO:3, SEQ ID NO:5 SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) that hybridizes under high stringency conditions with a nucleic acid molecule of any of b1) to b3); or
c) a vector comprising a nucleic acid molecule according to any one of b1) to b4); or
d) a host cell transformed with the vector of c); or
e) a ligand:
1) that binds specifically to the MAM domain containing polypeptide of any of a1) to a19); or
2) that binds specifically to the MAM domain containing polypeptide of any of a1) to a19) and is an antibody; or
f) a compound:
1) that increases the level of expression or activity of a polypeptide according to any of a1) to a19); or
2) that decreases the level of expression or activity of a polypeptide according to any of a1) to a19); or
g) a compound that binds to a polypeptide according to any of a1) to a19) without inducing any of the biological effects of the polypeptide; or
h) a compound that binds to a polypeptide according to any of a1) to a19) without inducing any of the biological effects of the polypeptide, wherein the compound is a natural or modified substrate, ligand, enzyme, receptor, or structural or functional mimetic; or
i) a pharmaceutical composition comprising any one of a) to h), and a pharmaceutically acceptable carrier; or
j) the pharmaceutical composition of i), further comprising one or more of cyclophosphamide (CTX), CLTA4-Ig, rapamycin, mycophenolic acid, methylprednisone, FTY720, an LFA-1 blocking agent, ICOS-Ig, an anti-apoptotic agent, an antagonist of cytolytic T cell function, TACI-Ig or methotrexate; or
k) a vaccine composition comprising any polypeptide of a1) to a19) or any nucleic acid molecule of b1) to b4); or
l) a kit useful for diagnosing disease, comprising a first container containing a nucleic acid probe that hybridizes under stringent conditions with a nucleic acid molecule of any one of b 1) to b4), a second container containing primers useful for amplifying the nucleic acid molecule, and instructions for using the probe and primers for facilitating the diagnosis of disease; or
m) a kit useful for diagnosing disease, comprising a first container containing a nucleic acid probe that hybridizes under stringent conditions with a nucleic acid molecule of any one of b1) to b4); a second container containing primers useful for amplifying the nucleic acid molecule; a third container holding an agent for digesting unhybridized RNA; and instructions for using the probe and primers for facilitating the diagnosis of disease; or
n) a kit comprising an array of nucleic acid molecules, at least one of which is a nucleic acid molecule according to any one of b1) to b4); or
o) a kit comprising one or more antibodies that bind to a polypeptide as recited in any one of a1) to a19); and a reagent useful for the detection of a binding reaction between the one or more antibodies and the polypeptide; or
p) a transgenic or knockout non-human animal that has been transformed to express higher, lower, or absent levels of a polypeptide according to any one of a1) to a19).

58. A method of using a composition of matter, comprising obtaining a composition of matter according to claim 57 and using said composition of matter in a method selected from the group consisting of: diagnosing a disease in a patient; treatment of a disease in a patient; monitoring the therapeutic treatment of a disease in a patient; identification of a compound that is effective in the treatment and/or diagnosis of a disease; and screening candidate compounds for a compound effective to treat a disease.

59. The method of claim 58, wherein said method of using a composition of matter comprises the method for treatment of a disease, comprising administering to the patient:
a) an isolated polypeptide selected from the group consisting of:
1) an amino acid sequence comprising SEQ ID NO:1;
2) an amino acid sequence comprising SEQ ID NO:2;
3) a fragment of said amino acid sequence which functions as a secreted protein or has an antigenic determinant in common with the polypeptide of 1);
4) a functional equivalent of 1) or 2);
5) an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18:
6) a fragment of said amino acid sequence of 5) which functions as a secreted protein or has an antigenic determinant in common with the polypeptide of 5);
7) a functional equivalent of 5);
8) a functional equivalent of 4) or 7), characterized in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; and is a LDL and/or MAM domain containing polypeptide;
9) the functional equivalent of 4), 7), or 8), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
10) the functional equivalent of 4), 7), or 8), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
11) a functional equivalent of 4), 7), 8), 9), or 10), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
12) the fragment of 3) or 6), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
13) the fragment of 3) or 6), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
14) the fragment of 3), 12), or 13), wherein the fragment has an antigenic determinant in common with the polypeptide of 1) or 2), or with the polypeptide of 5) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
15) the polypeptide of any one of 1) to 7), further comprising a histidine tag;
16) the polypeptide of any one of 1) to 7) or 15), wherein the polypeptide comprises a signal peptide;
17) the polypeptide of 16), whose sequence is recited in SEQ ID NO:20, SEQ ID NO:22, SEQ ID NO:24, SEQ ID NO:26, and/or SEQ ID NO:28;
18) a fusion polypeptide comprising a polypeptide of any one of 1) to 17); and
19) the fusion polypeptide of 18), comprising SEQ ID NO:4, SEQ ID NO:6, or SEQ ID NO:22; or
b) a purified nucleic acid molecule:
1) comprising a nucleic acid sequence encoding a polypeptide of any one of a1) to a19); or
2) comprising a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consisting of a nucleic acid sequence selected from the group consisting of SEQ ID NO:1, SEQ ID NO:3, SEQ ID NO:5, SEQ ID NO:7, SEQ ID NO:9, SEQ ID NO:1, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) that hybridizes under high stringency conditions with a nucleic acid molecule of any of b1) to b3); or
c) a vector comprising a nucleic acid molecule according to any one of b1) to b4); or
d) a host cell transformed with the vector of c); or
e) a ligand:
1) that binds specifically to the MAM domain containing polypeptide of any of a1) to a19); or
2) that binds specifically to the MAM domain containing polypeptide of any of a1) to a19) and is an antibody; or
f) a compound:
1) that increases the level of expression or activity of a polypeptide according to any of a1) to a19); or
2) that decreases the level of expression or activity of a polypeptide according to any of a1) to a19); or
g) a compound that binds to a polypeptide according to any of a1) to a19) without inducing any of the biological effects of the polypeptide; or
h) a compound that binds to a polypeptide according to any of a1) to a 19) without inducing any of the biological effects of the polypeptide, wherein the compound is a natural or modified substrate, ligand, enzyme, receptor, or structural or functional mimetic; or
i) a pharmaceutical composition comprising any one of a) to h), and a pharmaceutically acceptable carrier; or
j) the pharmaceutical composition of i), further comprising one or more of cyclophosphamide (CTX), CLTA4-Ig, rapamycin, mycophenolic acid, methylprednisone, FTY720, an LFA-1 blocking agent, ICOS-Ig, an anti-apoptotic agent, an antagonist of cytolytic T cell function, TACI-Ig or methotrexate.

60. The method of claim 59, wherein the disease includes one or more of among reproductive disorders, including infertility, cell proliferative disorders, including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anemia, sepsis, shock, reperfusion injury, stroke and ischemia; neurological disorders including central nervous system disease, Alzheimer's disease, brain injury, amyotrophic lateral sclerosis, and pain; developmental disorders; metabolic disorders including diabetes mellitlis, osteoporosis, hypercholesterolemia, hyperlipidemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

61. The method of claim 59, wherein the disease includes one or more of among inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea.

62. The method of claim 59, wherein the disease is one in which MAM domain containing proteins and/or LDL domain containing proteins are implicated.

63. The method of claim 59, wherein the disease is one for which the expression of the natural gene or the activity of the polypeptide is lower in a diseased patient when compared to the level of expression or activity in a healthy patient, the polypeptide, nucleic acid molecule, vector, ligand, compound or composition administered to the patient is an agonist.

64. The method of claim 59, wherein the disease is one for which expression of the natural gene or activity of the polypeptide is higher in a diseased patient when compared to the level of expression or activity in a healthy patient, the polypeptide, nucleic acid molecule, vector, ligand, compound or composition administered to the patient is an antagonist.

65. The method of claim 58, wherein said method of using a composition of matter comprises the method for diagnosing a disease in a patient, comprising assessing the level of expression of a natural gene encoding a polypeptide, or assessing the activity of the polypeptide, in tissue from said patient; and comparing said level of expression or activity to a control level, wherein a level that is different to said control level is indicative of disease, and wherein the polypeptide:
a) has an amino acid sequence comprising SEQ ID NO:1; or
b) has an amino acid sequence comprising SEQ ID NO:2; or
c) is a fragment of said amino acid sequence which functions as a secreted protein or has an antigenic determinant in common with the polypeptide of a); or
d) is a functional equivalent of a) or b); or
e) has an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18; or
f) is a fragment of said amino acid sequence of e) which functions as a secreted protein or has an antigenic determinant in common with the polypeptide of e); or
g) is a functional equivalent of e); or
h) a functional equivalent of d) or g), characterized in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; and is a LDL and/or MAM domain containing polypeptide; or
i) the functional equivalent of d), g), or h), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
j) the functional equivalent of d), g), or h), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
k) a functional equivalent of d), g), h), i), or j), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or
l) the fragment of c) or f), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
m) the fragment of c) or f), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
n) the fragment of c), l), or m), wherein the fragment has an antigenic determinant in common with the polypeptide of a) or b), or with the polypeptide of f) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or
o) the polypeptide of any one of a) to g), further comprising a histidine tag; or
p) the polypeptide of any one of a) to g) or o), wherein the polypeptide comprises a signal peptide; or
q) the polypeptide of q), whose sequence is recited in SEQ ID NO:20, SEQ ID NO:22, SEQ ID NO:24, SEQ ID NO:26, and/or SEQ ID NO:28; or
r) a fusion polypeptide comprising a polypeptide of any one of a) to q); or
s) the fusion polypeptide of r), comprising SEQ ID NO:4, SEQ ID NO:6, or SEQ ID NO:22.

66. The method of claim 65, which is carried out in vitro.

67. The method of claim 65, comprising:
a) contacting a ligand with a biological sample under conditions suitable for the formation of a ligand-polypeptide complex; and
b) detecting said complex, wherein the ligand binds specifically to the polypeptide of any of a) to s) of claim 65, or wherein the ligand is an antibody that binds specifically to the polypeptide of any of a) to s) of claim 65.

68. The method of claim 65, comprising:
a) contacting a sample of tissue from the patient with a nucleic acid probe under stringent conditions that allow the formation of a hybrid complex between a nucleic acid molecule and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease, wherein the nucleic acid molecule:
1) comprises a nucleic acid sequence encoding a polypeptide of any of a) to s) of claim 65; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) hybridizes under high stringency conditions with a nucleic acid molecule of any of c1) to c3).

69. The method of claim 65, comprising:
a) contacting a sample of nucleic acid from tissue of the patient with a nucleic acid primer under stringent conditions that allow the formation of a hybrid complex between a nucleic acid molecule and the primer;
b) contacting a control sample with said primer under the same conditions used in step a);
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease, wherein the nucleic acid molecule:
1) comprises a nucleic acid sequence encoding a polypeptide of any of a) to s) of claim 65; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) hybridizes under high stringency conditions with a nucleic acid molecule of any of d1) to d3).

70. The method of claim 65, comprising:
a) obtaining a tissue sample from a patient being tested for disease;
b) isolating a nucleic acid molecule from said tissue sample; and
c) diagnosing the patient for disease by detecting the presence of a mutation which is associated with disease in the nucleic acid molecule as an indication of the disease, wherein the nucleic acid molecule:
1) comprises a nucleic acid sequence encoding a polypeptide of any of a) to s) of claim 65; or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) hybridizes under high stringency conditions with a nucleic acid molecule of any of c1) to c3).

71. The method of claim 70, further comprising amplifying the nucleic acid molecule to form an amplified product and detecting the presence or absence of a mutation in the amplified product.

72. The method of claim 70, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

73. The method of claim 65, wherein the disease is one or more of among reproductive disorders, including infertility, cell proliferative disorders including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anemia, sepsis, shock, reperfusion injury, stroke and ischemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolemia, hyperlipidemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

74. The method of claim 65, wherein the disease is one or more of among inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea.

75. The method of claim 65, wherein the disease is a disease in which MAM domain containing proteins and/or LDL domain containing proteins are implicated.

76. The method of claim 58, wherein said method of using a composition of matter comprises the method of monitoring the therapeutic treatment of a disease, comprising monitoring over a period of time the level of expression or activity of a polypeptide, or the level of expression of a nucleic acid molecule, in tissue from said patient, wherein altering said level of expression or activity over the period of time towards a control level is indicative of regression of said disease, wherein
a) the polypeptide is selected from the group consisting of:
1) an amino acid sequence comprising SEQ ID NO:1;
2) an amino acid sequence comprising SEQ ID NO:2;
3) a fragment of said amino acid sequence which functions as a secreted protein or has an antigenic determinant in common with the polypeptide of 1);
4) a functional equivalent of 1) or 2);
5) an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18;
6) a fragment of said amino acid sequence of 5) which functions as a secreted protein or has an antigenic determinant in common with the polypeptide of 5);
7) a functional equivalent of 5);
8) a functional equivalent of 4) or 7), characterized in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; and is a LDL and/or MAM domain containing polypeptide;
9) the functional equivalent of 4), 7), or 8), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
10) the functional equivalent of 4), 7), or 8), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
11) a functional equivalent of 4), 7), 8), 9), or 10), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
12) the fragment of 3) or 6), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
13) the fragment of 3) or 6), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
14) the fragment of 3), 12), or 13), wherein the fragment has an antigenic determinant in common with the polypeptide of 1) or 2), or with the polypeptide of 5) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
15) the polypeptide of any one of 1) to 7), further comprising a histidine tag;
16) the polypeptide of any one of 1) to 7) or 15), wherein the polypeptide comprises a signal peptide;
17) the polypeptide of 16), whose sequence is recited in SEQ ID NO:20, SEQ ID NO:22, SEQ ID NO:24, SEQ ID NO:26, and/or SEQ ID NO:28;
18) a fusion polypeptide comprising a polypeptide of any one of 1) to 17); and
19) the fusion polypeptide of 18), comprising SEQ ID NO:4, SEQ ID NO:6, or SEQ ID NO:22; and wherein
b) the purified nucleic acid molecule:
1) comprises a nucleic acid sequence encoding a polypeptide of any one of a1) to a19); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) hybridizes under high stringency conditions with a nucleic acid molecule of any of b1) to b3).

77. The method of claim 76, wherein the disease is one or more of among reproductive disorders, including infertility, cell proliferative disorders including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anemia, sepsis, shock, reperfusion injury, stroke and ischemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolemia, hyperlipidemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

78. The method of claim 76, wherein the disease is one or more of among inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea.

79. The method of claim 76, wherein the disease is a disease in which MAM domain containing proteins and/or LDL domain containing proteins are implicated.

80. The method of claim 58, wherein said method of using a composition of matter comprises the method for identification of a compound that is effective in the treatment and/or diagnosis of a disease, comprising contacting a polypeptide or a nucleic acid molecule with one or more compounds suspected of possessing binding affinity for said polypeptide or nucleic acid molecule, and selecting a compound that binds specifically to said nucleic acid molecule or polypeptide, wherein
a) the polypeptide is selected from the group consisting of:
1) an amino acid sequence comprising SEQ ID NO:1;
2) an amino acid sequence comprising SEQ ID NO:2;
3) a fragment of said amino acid sequence which functions as a secreted protein or has an antigenic determinant in common with the polypeptide of 1);
4) a functional equivalent of 1) or 2);
5) an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18;
6) a fragment of said amino acid sequence of 5) which functions as a secreted protein or has an antigenic determinant in common with the polypeptide of 5);
7) a functional equivalent of 5);
8) a functional equivalent of 4) or 7), characterized in that it is homologous to an amino acid sequence selected from the group consisting of SEQ ID NO:2, SEQ ID NO:4, SEQ ID NO:6, SEQ ID NO:8, SEQ ID NO: 10, SEQ ID NO:12, SEQ ID NO:14, SEQ ID NO:16, and SEQ ID NO:18; and is a LDL and/or MAM domain containing polypeptide;
9) the functional equivalent of 4), 7), or 8), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
10) the functional equivalent of 4), 7), or 8), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
11) a functional equivalent of 4), 7), 8), 9), or 10), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
12) the fragment of 3) or 6), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
13) the fragment of 3) or 6), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing;
14) the fragment of 3), 12), or 13), wherein the fragment has an antigenic determinant in common with the polypeptide of 1) or 2), or with the polypeptide of 5) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18;
15) the polypeptide of any one of 1) to 7), further comprising a histidine tag;
16) the polypeptide of any one of 1) to 7) or 15), wherein the polypeptide comprises a signal peptide;
17) the polypeptide of 16), whose sequence is recited in SEQ ID NO:20, SEQ ID NO:22, SEQ ID NO:24, SEQ ID NO:26, and/or SEQ ID NO:28;
18) a fusion polypeptide comprising a polypeptide of any one of 1) to 17); and
19) the fusion polypeptide of 18), comprising SEQ ID NO:4, SEQ ID NO:6, or SEQ ID NO:22; and wherein
b) the purified nucleic acid molecule:
1) comprises a nucleic acid sequence encoding a polypeptide of any one of a1) to a19); or
2) comprises a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
3) consists of a nucleic acid sequence selected from the group consisting of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, and SEQ ID NO:27, or is a redundant equivalent or fragment of any of the foregoing; or
4) hybridizes under high stringency conditions with a nucleic acid molecule of any of b1) to b3).

81. The method of claim 80, wherein the disease is one or more of among reproductive disorders, including infertility, cell proliferative disorders including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anemia, sepsis, shock, reperfusion injury, stroke and ischemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolemia, hyperlipidemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

82. The method of claim 80, wherein the disease is one or more of among inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea.

83. The method of claim 80, wherein the disease is a disease in which MAM domain containing proteins and/or LDL domain containing proteins are implicated.

84. The method of claim 58, wherein said method of using a composition of matter comprises the method for screening candidate compounds, comprising contacting a non-human transgenic animal with a candidate compound and determining the effect of the compound on the disease of the transgenic animal, wherein the transgenic animal has been transformed to express higher, lower, or absent levels of a polypeptide, wherein the polypeptide.
a) has an amino acid sequence comprising SEQ ID NO:1; or
b) has an amino acid sequence comprising SEQ ID NO:2; or
c) is a fragment of said amino acid sequence which functions as a secreted protein or has an antigenic determinant in common with the polypeptide of a); or
d) is a functional equivalent of a) or b); or
e) has an amino acid sequence selected from the group consisting of SEQ ID NO:6, SEQ ID NO:8, SEQ ID NO:10, SEQ ID NO:12, SEQ ID NO:14, SEQ ID NO:16, and SEQ ID NO:18; or
f) is a fragment of said amino acid sequence of e) which functions as a secreted protein or has an antigenic determinant in common with the polypeptide of e); or
g) is a functional equivalent of e); or
h) a functional equivalent of d) or g), characterized in that it is homologous to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; and is a LDL and/or MAM domain containing polypeptide; or
i) the functional equivalent of d), g), or h), wherein the functional equivalent has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
j) the functional equivalent of d), g), or h), wherein the functional equivalent has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO: 10, SEQ ID NO:12, SEQ ID NO:14, SEQ ID NO:16, and SEQ ID NO:18; or with an active fragment of any of the foregoing; or
k) a functional equivalent of d), g), h), i), or j), wherein the functional equivalent exhibits significant structural homology with a polypeptide having the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or
l) the fragment of c) or f), wherein the fragment has greater than 50% sequence identity to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
m) the fragment of c) or f), wherein the fragment has greater than 60%, 70%, 80%, 90%, 95%, 98%, or 99% sequence identity with an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or with an active fragment of any of the foregoing; or
n) the fragment of c), l), or m), wherein the fragment has an antigenic determinant in common with the polypeptide of a) or b), or with the polypeptide of f) which consists of 7 or more amino acid residues from the amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18; or
o) the polypeptide of any one of a) to g), further comprising a histidine tag; or
p) the polypeptide of any one of a) to g) or o), wherein the polypeptide comprises a signal peptide; or
q) the polypeptide of q), whose sequence is recited in SEQ ID NO:20, SEQ ID NO:22, SEQ ID NO:24, SEQ ID NO:26, and/or SEQ ID NO:28; or
r) a fusion polypeptide comprising a polypeptide of any one of a) to q); or
s) the fusion polypeptide of r), comprising SEQ ID NO:4, SEQ ID NO:6, or SEQ ID NO:22.

85. The method of claim 84, wherein the disease is one or more of among reproductive disorders, including infertility, cell proliferative disorders including neoplasm, melanoma, lung, colorectal, breast, pancreas, head and neck and other solid tumors; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, anemia, sepsis, shock, reperfusion injury, stroke and ischemia; neurological disorders including central nervous system disease, Alzheimer's disease, brain injury, amyotrophic lateral sclerosis, and pain; developmental disorders; metabolic disorders including diabetes mellitus, osteoporosis, hypercholesterolemia, hyperlipidemia, and obesity; gastrointestinal disorders such as Crohn's disease, disorders of lipid metabolism and anorexia; gout; Grave's disease; AIDS and renal disease; prostatitis; infections including viral infection, bacterial infection, fungal infection and parasitic infection, pathological conditions associated with the placenta and other pathological conditions.

86. The method of claim 84, wherein the disease is one or more of among inflammatory diseases, autoimmune diseases, CTLA4-related disease, diseases of the small intestine, Crohn's disease, small bowel disease, inflammatory bowel disease, lupus, celiac disease, Whipple's disease, neoplasias or diarrhea.

87. The method of claim 84, wherein the disease is a disease in which MAM domain containing proteins and/or LDL domain containing proteins are implicated.

88. A method of selecting biologically active compounds comprising:
(i) contacting a candidate compound with recombinant host cells expressing an INSP152 polypeptide; and
(ii) selecting compounds that bind said INSP152 polypeptide at the surface of said cells and/or that modulate the activity of the INSP152 polypeptide.

89. An isolated polypeptide comprising an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, and SEQ ID NO:18.


SEQUENCE LISTING (42 sequences)

SEQ ID NO: 1 - DNA, 531 bp, Homo sapiens
cagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggc

SEQ ID NO: 2 - protein, 177 aa, Homo sapiens
QQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSG

SEQ ID NO: 3 - DNA, 569 bp, Homo sapiens
cagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatat

SEQ ID NO: 4 - protein, 189 aa, Homo sapiens
QQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDD

SEQ ID NO: 5 - DNA, 598 bp, Homo sapiens
ttgcctgtgttttcaattctacactggctcagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatat

SEQ ID NO: 6 - protein, 198 aa, Homo sapiens
ACVFNSTLAQQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDD

SEQ ID NO: 7 - DNA, 5997 bp, Homo sapiens
cagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataataca

SEQ ID NO: 8 - protein, 1999 aa, Homo sapiens
QQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNT

SEQ ID NO: 9 - DNA, 6051 bp, Homo sapiens
cagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataataca

SEQ ID NO: 10 - protein, 2017 aa, Homo sapiens
QQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNT

SEQ ID NO: 11 - DNA, 6326 bp, Homo sapiens
ttgcctgtgttttcaattctacactggctcagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaa

SEQ ID NO: 12 - protein, 2026 aa, Homo sapiens
ACVFNSTLAQQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNT

SEQ ID NO: 13 - DNA, 6243 bp, Homo sapiens
cagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaa

SEQ ID NO: 14 - protein, 2081 aa, Homo sapiens
QQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNTWTLLGIGLAFLMTHITVAVLCFLANRKVPIRKTEGSGNCAFVNPVYGNWSNPEKTESSVYSFSNPLYGTTSGSLETLSHHLK

SEQ ID NO: 15 - DNA, 6297 bp, Homo sapiens
cagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaa

SEQ ID NO: 16 - protein, 2099 aa, Homo sapiens
QQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNTWTLLGIGLAFLMTHITVAVLCFLANRKVPIRKTEGSGNCAFVNPVYGNWSNPEKTESSVYSFSNPLYGTTSGSLETLSHHLK

SEQ ID NO: 17 - DNA, 6326 bp, Homo sapiens; identical to SEQ ID NO: 11

SEQ ID NO: 18 - protein, 2108 aa, Homo sapiens
ACVFNSTLAQQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNTWTLLGIGLAFLMTHITVAVLCFLANRKVPIRKTEGSGNCAFVNPVYGNWSNPEKTESSVYSFSNPLYGTTSGSLETLSHHLK

SEQ ID NO: 19 - DNA, 588 bp, Homo sapiens
atgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggc

SEQ ID NO: 20 - protein, 196 aa, Homo sapiens
MNETFCCLWIACVFNSTLAQQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSG

SEQ ID NO: 21 - DNA, 626 bp, Homo sapiens
atgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatat

SEQ ID NO: 22 - protein, 208 aa, Homo sapiens
MNETFCCLWIACVFNSTLAQQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDD

SEQ ID NO: 23 - DNA, 6054 bp, Homo sapiens
atgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataataca

SEQ ID NO: 24 - protein, 2018 aa, Homo sapiens
MNETFCCLWIACVFNSTLAQQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNT

SEQ ID NO: 25 - DNA, 6300 bp, Homo sapiens
atgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaa

SEQ ID NO: 26 - protein, 2100 aa, Homo sapiens
MNETFCCLWIACVFNSTLAQQGTESFQCDNGVSLPPDSICDFTDQCGDSSDERHWLSFAHPCNPLGSSLESDNWNKKKLESGACLGFGAAAASQDPRQELRKKLVWTADPRNWRAITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNTWTLLGIGLAFLMTHITVAVLCFLANRKVPIRKTEGSGNCAFVNPVYGNWSNPEKTESSVYSFSNPLYGTTSGSLETLSHHLK

SEQ ID NO: 27 - DNA, 6354 bp, Homo sapiens
atgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactgtttgaattatgaaagatgtgattttgaggatggtctctgtcatatgactcaagatcagagtctgcaacctagttggacaaagagaagtgggatgattggtctatcacctccattttatgatcacaatggtgatgtgtctgctcacttcctctcactggtttccagagtggattctatttcctcaagtttaagaagcagagttttccttccaacaaatgatcaacatgactgccagattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaa

SEQ ID NO: 28 - protein, 2118 aa, Homo sapiens
MNETFCCLWIACVFNSTLAQQGTESFQCDNGVSLPPDSICDFTDQCGDSSDERHCLNYERCDFEDGLCHMTQDQSLQPSWTKRSGMIGLSPPFYDHNGDVSAHFLSLVSRVDSISSSLRSRVFLPTNDQHDCQITFYYFSCQVSGKLMVGLQTACGGPIQHLWQNTAALPNQWERNVIKIQSSQRFQVVFEGQMASTYEQDEVIAIDDISFSSGCLPANDGILLCQEALNAERELCHPDTDLCRFDATDEELRLCQACGFEFDMCEWTSEASAGQISWMRTKAREIPAFESTPQQDQGGDDEGYYVWVGAKHGFTLNHLDSRAYLNSSVCHCLGKSCHLQFYYAMESSVLRVRLYNNKEEEIFWTYNISTHSQWVKADVLIPEDLKTFKTQSRKLCSADEFPCTSGQCIAKESVCDSRQDCSDESDEDPATCSKHLTCDFESGFCGWEPFLTEDSHWKLMKGLNNGEHHFPAADHTANINHGSFIYLEAQRSPGVAKLGSPVLTKLLTASTPCQVQFWYHLSQHSNLSVFTRTSLDGNLQKQGKIIRFSESQWSHAKIDLIAEAGESTLPFQLILEATVLSSNATVALDDISVSQECEISYKSLPRTSTQSKFSKCDFEANSCDWFEAISGDHFDWIRSSQSELSADFEHQAPPRDHSLNASQGHFMFILKKSSSLWQVAKLQSPTFSQTGPGCILSFWFYNYGLSVGAAELQLHMENSHDSTVIWRVLYNQGKQWLEATIQLGRLSQPFHLSLDKVSLGIYDGVSAIDDIRFENCTLPLPAESCEGLDHFWCRHTRACIEKLRLCDLVDDCGDRTDEVNCAPELQCNFETGICNWEQDAKDDFDWTRSQGPTPTLNTGPMKDNTLGTAKGHYLYIESSEPQAFQDSAALLSPILNATDTKGCTFRFYYHMFGKRIYRLAIYQRIWSDSRGQLLWQIFGNQGNRWIRKHLNISSRQPFQILVEASVGDGFTGDIAIDDLSFMDCTLYPGNLPADLPTPPETSVPVTLPPHNCTDNEFICRSDGHCIEKMQKCDFKYDCPDKSDEASCVMEVCSFEKRSLCKWYQPIPVHLLQDSNTFRWGLGNGISIHHGEENHRPSVDHTQNTTDGWYLYADSSNGKFGDTADILTPIISLTGPKCTLVFWTHMNGATVGSLQVLIKKDNVTSKLWAQTGQQGAQWKRAEVFLGIRSHTQIVFRAKRGISYIGDVAVDDISFQDCSPLLSPERKCTDHEFMCANKHCIAKDKLCDFVNDCADNSDETTFICRTSSGRCDFEFDLCSWKQEKDEDFDWNLKASSIPAAGTEPAADHTLGNSSGHYIFIKSLFPQQPMRAARISSPVISKRSKNCKIIFHYHMYGNGIGALTLMQVSVTNQTKVLLNLTVEQGNFWRREELSLFGDEDFQLKFEGRVGKGQRGDIALDDIVLTENCLSLHDSVQEELAVPLPTGFCPLGYRECHNGKCYRLEQSCNFVDNCGDNTDENECGSSCTFEKGWCGWQNSQADNFDWVLGVGSHQSLRPPKDHTLGNENGHFMYLEATAVGLRGDKAHFRSTMWRESSAACTMSFWYFVSAKATGSIQILIKTEKGLSKVWQESKQNPGNHWQKADILLGKLRNFEVIFQGIRTRDLGGGAAIDDIEFKNCTTVGEISELCPEITDFLCRDKKCIASHLLCDYKPDCSDRSDEAHCAHYTSTTGSCNFETSSGNWTTACSLTQDSEDDLDWAIGSRIPAKALIPDSDHTPGSGQHFLYVNSSGSKEGSVARITTSKSFPASLGMCTVRFWFYMIDPRSMGILKVYTIEESGLNILVWSVIGNKRTGWTYGSVPLSSNSPFKVAFEADLDGNEDIFIALDDISFTPECVTGGPVPVQPSPCEADQFSCIYTLQCVPLSGKCDGHEDCIDGSDEMDCPLSPTPPLCSNMEFPCSTDECIPSLLLCDGVPDCHFNEDELICSNKSCSNGALVCASSNSCIPAHQRCDGFADCMDFQLDESSCSECPLNYCRNGGTCVVEKNGPMCRCRQGWKGNRCHIKFNPPATDFTYAQNNTWTLLGIGLAFLMTHITVAVLCFLANRKVPIRKTEGSGNCAFVNPVYGNWSNPEKTESSVYSFSNPLYGTTSGSLETLSHHLK

SEQ ID NO: 29 - protein, 13 aa, Artificial Sequence
  Fusion protein linker sequence
EFGAGLVLGGQFM

SEQ ID NO: 30 - DNA, 27 bp, Artificial Sequence
  Primer INSP152-CP1
atgaatgaaactttttgttgcctttgg

SEQ ID NO: 31 - DNA, 22 bp, Artificial Sequence
  Primer INSP152-CP2
gcaagcagcctgaactgaaaga

SEQ ID NO: 32 - DNA, 23 bp, Artificial Sequence
  Primer INSP152-CP3
attgcctgtgttttcaattctac

SEQ ID NO: 33 - DNA, 24 bp, Artificial Sequence
  Primer INSP152-CP4
tatatcatcaatagcaatgacttc

SEQ ID NO: 34 - DNA, 19 bp, Artificial Sequence
  Primer T7
taatacgactcactatagg

SEQ ID NO: 35 - DNA, 18 bp, Artificial Sequence
  Primer T3
attaaccctcactaaagg

SEQ ID NO: 36 - DNA, 24 bp, Artificial Sequence
  Primer h-INSP152-D1-SV1-519F
ggagagaaatgtcatcaaaatcca

SEQ ID NO: 37 - DNA, 23 bp, Artificial Sequence
  Primer h-INSP152-D1-SV1-584R
gccatttgaccttcaaaaacaac

SEQ ID NO: 38 - DNA, 18 bp, Artificial Sequence
  Primer hGAPDH-F
ccacccatggcaaattcc

SEQ ID NO: 39 - DNA, 22 bp, Artificial Sequence
  Primer hGAPDH-R
gatgggatttccattgatgaca

SEQ ID NO: 40 - DNA, 21 bp, Artificial Sequence
  Primer Intron-hGAPDH-F
cctagtcccagggctttgatt

SEQ ID NO: 41 - DNA, 21 bp, Artificial Sequence
  Primer Intron-hGAPDH-R
ctgtgctcccactcctgattt

SEQ ID NO: 42 - DNA, 6500 bp, Homo sapiens
gaatgtttccaatgatggacttacttattacaactgcttgatctctaatagacaataccaagtaatgctcttcttcctggacagaatgttggcattccccatgaatgaaactttttgttgcctttggattgcctgtgttttcaattctacactggctcagcaagggacagaaagctttcagtgtgacaatggagtctccttgcctcctgacagcatttgtgacttcacagatcagtgtggggatagcagtgatgaacggcactggctttcatttgctcatccttgcaatccattggggtctagtttggaatctgacaactggaacaaaaagaaacttgaatccggtgcatgccttggttttggtgctgctgctgcttctcaagatcctcggcaggaattaagaaagaagctggtgtggacagcagatccccgaaattggcgggctattacattttattacttctcctgccaagtgagtggcaaattaatggttgggcttcaaactgcatgtggaggtcctattcagcatttatggcaaaacacagctgcactcccaaatcagtgggagagaaatgtcatcaaaatccagagttcacagagatttcaggttgtttttgaaggtcaaatggcttcaacgtatgaacaggatgaagtcattgctattgatgatatatctttcagttcaggctgcttgcctgccaatgatggaattttactgtgtcaagaagcattgaatgctgagcgggagctatgccatccagatacagatctctgcagatttgatgctacagatgaagagttgagattgtgtcaggcctgtgggtttgaatttgacatgtgtgagtggacgtcagaagcatctgctggccaaatttcctggatgcgcacaaaagcgagagagatccctgcattcgaatccacacctcagcaggatcaaggaggtgatgatgaaggttattatgtatgggtaggcgctaagcatggtttcactcttaaccatttagacagcagggcttacctaaatagctctgtgtgtcattgcctgggcaagagctgtcatcttcaattctattatgcaatggaaagcagtgtcctgagagtaagactgtataataataaggaagaagaaatattttggacatacaacatatcaactcacagccaatgggtgaaagcagatgtgttaataccagaagatctgaagacatttaagacccaatccagaaagctttgctctgcagacgaattcccttgcactagtggccagtgcatcgccaaagaatctgtctgtgactctcggcaggactgctccgatgagagtgatgaagacccagcaacttgctcaaagcatctcacctgtgactttgagtcgggtttctgcggttgggagccatttctcacagaagattcacactggaagctgatgaaaggattgaataatggagagcaccactttcctgcagctgatcacacagcaaacataaatcatggatcgtttatttatttggaggcacagcgctcccccggggtggccaagcttggaagtcctgttcttacaaaattgctcactgcctctaccccatgtcaggtgcagttttggtatcatttgtctcaacattcaaatctctcagtttttacaagaacgtctctagatggaaacttgcaaaagcagggcaaaataatcagattctccgaatctcagtggagccacgcaaaaattgatctcattgcagaagcgggagaatctactctaccttttcagttaattttggaagctactgttttgtcgtcaaatgctaccgttgctctagatgacatcagtgtgtcccaggaatgtgaaatttcctataaatcactaccaaggaccagtacacaaagcaagttttccaagtgtgactttgaagcaaacagctgtgattggtttgaagcaattagtggtgaccattttgactggatacggagctctcagagtgaactttctgctgattttgagcaccaggctccacctcgggatcatagtctcaacgcatctcaagggcattttatgttcattctgaagaaaagcagcagcttgtggcaagttgctaagcttcagagcccaactttcagccagacaggacctggatgcatactttccttctggttctataactatggcctgtcagtgggagcagctgagctgcagctacatatggaaaattctcatgactcaacagtgatttggagagtattatacaatcagggcaaacaatggttggaggcaaccattcagctagggcgcctttcgcagcccttccatttgtcactagataaagtcagtctgggcatttatgatggggtctcagctattgatgacatccgatttgaaaattgtactctccctcttcctgctgagagctgtgaagggctggatcatttctggtgtcgccacaccagggcttgcatagaaaagcttcggttatgtgatctggtggatgactgtggtgatcgtactgatgaagtcaactgtgcacctgagctgcagtgtaactttgaaactggaatctgtaactgggaacaagatgcaaaagatgactttgattggaccaggagccagggtccaactccaacacttaacacagggccaatgaaagataacactctgggcacagctaaaggacactatctctacatagaatcttcagagccacaggcttttcaagacagtgctgccttactcagcccaatccttaatgccactgatacaaaaggctgcaccttccgcttctattaccacatgtttggaaagcgcatttataggttggcaatctaccaacgaatctggagtgactcaaggggacagctgctgtggcagatatttgggaatcaaggcaacagatggattaggaaacacctcaacatttccagcaggcagccctttcagatattggtggaggcttcagtgggagatggcttcactggagatattgcgattgatgatctgtcatttatggactgcaccctctaccctggtaatttgccagcagacctcccaactccaccagaaacgtcagttcctgtaacattacctccacacaactgcacagacaatgaatttatctgcaggtctgatggtcactgcattgaaaaaatgcagaaatgtgattttaaatatgactgccctgacaaatcagatgaagcatcctgtgttatggaagtttgcagctttgagaaaagaagcctgtgtaaatggtatcaaccaatcccagtacatttgcttcaagattcaaacacattcaggtgggggcttgggaacgggatcagcattcatcatggggaagaaaaccacaggccatcagtggatcatacacaaaataccactgatggctggtacctgtatgctgacagttctaatgggaaatttggtgacacggctgacattctcactcctatcatttcactcacgggaccaaaatgtaccttggtgttctggacacatatgaatggggccaccgttggttctctccaggtgctcatcaagaaagataacgttacttctaaattgtgggctcaaactggacagcaaggtgcacagtggaagagagcagaagtgtttttaggcattcgttcacatacacagattgtcttcagagccaaacgtggtatcagttacataggagatgtagcagtggatgatatttccttccaagattgctcccctttgcttagcccagagagaaagtgtactgatcatgaattcatgtgtgctaataagcactgcattgccaaagacaagctgtgtgattttgtgaatgattgtgctgataattcagatgagactactttcatttgccgtacctccagtgggcgctgtgatttcgaatttgatctttgttcctggaagcaggagaaagatgaggactttgactggaacctgaaagctagcagcatccctgcagcaggcacagagccagcagcagatcacactttgggaaattcatctggtcattacatctttataaagagtttgtttcctcagcagcccatgagagctgccagaatttcaagtccagttataagtaagagaagcaaaaactgcaagattatttttcattatcacatgtatggaaatggcattggggcactcaccttaatgcaggtgtcagtcacaaaccaaacgaaggttctacttaacctcactgtagaacaaggcaatttctggcggagagaagaactgtcactgtttggtgatgaagacttccaactcaaatttgaaggtagagttgggaaaggtcagcgtggagacattgcacttgatgacattgtgcttacagaaaattgtctatcactccatgattccgtgcaagaagaactggcagtgcctcttccaacaggtttctgcccacttggctatagggaatgtcataatggaaaatgctataggctggaacaaagctgtaacttcgtagataactgtggagataatactgatgaaaatgagtgtggtagctcctgtacttttgaaaaaggctggtgtggctggcaaaactcccaggctgacaactttgattgggttttaggggttggctctcatcaaagcttaagacctcccaaagaccacacacttggaaatgaaaatgggcacttcatgtatctggaagctactgcagtgggccttcggggtgacaaagcacacttcaggagtaccatgtggcgagaatccagtgcagcctgcaccatgagcttctggtatttcgtatctgcaaaggccacaggatccattcagattctcatcaagacagagaaaggactatcaaaagtatggcaagaaagtaagcagaaccctggtaatcattggcaaaaggctgacatcctgctaggaaagttaaggaattttgaagtcatatttcaaggtatcagaacaagggacctgggaggaggagctgcaattgatgatattgaatttaaaaactgcacaactgtgggagagatctctgagctttgtccggaaatcactgattttttgtgccgggacaagaagtgcattgcatcccaccttctttgtgactataagccagactgctctgataggtctgatgaagctcactgtgcacattatacaagcacaacaggaagctgcaattttgaaacaagttcaggaaactggaccacagcctgcagtcttactcaagactctgaggatgacttggactgggccattggcagcagaattcctgccaaagcattaattccagactctgatcacacgccaggtagtggtcagcacttcctgtacgtcaactcatctggctccaaggaaggatccgttgccagaattactacttccaaatccttcccagcaagccttggaatgtgtactgttcggttctggttctacatgattgatcccaggagtatgggaatattaaaggtgtataccattgaagaatcggggctaaacatcctggtgtggtcagtgattggaaataaaagaacgggatggacatatggctctgtgcctctctccagtaacagtccgtttaaggtggcatttgaagctgatttggatggaaatgaggacatctttattgctcttgatgacatctcttttaccccagagtgtgtgactggaggtcctgtcccagtgcagccatcaccctgtgaagctgatcagttttcttgtatctacacactccaatgtgtccctctctcagggaaatgtgatggacatgaagactgcatagatggatctgatgaaatggattgtcctctcagccccacccctccactctgtagtaacatggagttcccgtgctctacagacgagtgtataccttccctcctgctatgcgatggagtgcccgactgccactttaatgaagatgagctcatctgctccaacaaaagctgttctaatggagctctggtgtgtgcctcctccaacagctgtatcccagcccaccagcgctgtgatggttttgccgactgcatggatttccagcttgatgagtccagctgctccgaatgtccattaaattactgcagaaatggtgggacttgtgtagtggagaaaaatggtcctatgtgtcgatgtagacaaggctggaaaggaaatcgatgccatatcaagtttaatcctcctgctacagacttcacatacgctcagaataatacatggactctcctgggtattggattagcattcctgatgactcacatcacagttgcagtcttgtgttttcttgcaaacagaaaggtaccaataaggaaaaccgagggaagtggtaactgtgcctttgtcaatccagtttacgggaactggagcaacccagagaaaacagagagttctgtctattccttctcaaacccattatatggcacaacatcaggaagcctggagaccctgtcacatcatctcaaatagcagcatcgagaccaagtctgatccaacatgtgtagtttctagaaaattgaagtctccacaatctgatagaaactcatcttctacaatggtaaaaaga